(19) United States
(12) Patent Application Publication
(10) Pub. No.: US 2021/0361575 A1
(43) Pub. Date: Nov. 25, 2021

(54) LIPID PRODRUGS FOR USE IN DRUG DELIVERY

(71) Applicant: NEW MEXICO TECH UNIVERSITY RESEARCH PARK CORPORATION, Socorro, NM (US)

(72) Inventors: Michaelann TARTIS, Socorro, NM (US); Liliya FROLOVA, Socorro, NM (US)

(21) Appl. No.: 17/046,270

(22) PCT Filed: Apr. 11, 2019
(86) PCT No.: PCT/US19/26902

§ 371 Date: Oct. 8, 2020

Related U.S. Application Data
(60) Provisional application No. 62/656,035, filed on Apr. 11, 2018.

(51) Int. Cl. A61K 9/127; A61K 47/02; A61K 47/24; A61K 31/4745; A61K 31/7068; A61K 31/365; A61K 31/4741; A61K 47/10; A61K 47/26; A61K 47/54

(52) U.S. Cl. CPC A61K 9/1271; A61K 47/02; A61K 47/24; A61K 31/4745; A61K 47/544; A61K 31/365; A61K 31/4741; A61K 47/10; A61K 47/26; A61K 31/7068

(57) ABSTRACT

The present disclosure describes the synthesis and use of lipid prodrugs that self-assemble into lipid microbubbles or liposomes. The prodrug-loaded microbubbles or liposomes can be activated intracellularly using an external stimulus, for example, using ultrasound waves.

### CROSS REFERENCE

This application claims the benefit of U.S. Provisional Application No. 62/656,035, filed on Apr. 11, 2018, which is incorporated herein by reference in its entirety.

### GOVERNMENT RIGHTS

This invention was made with the support of the United States government under Grant Numbers P20 GM103451 and P20 RR016480 by the National Institutes of Health.

### BACKGROUND

Natural and synthetic chemotherapy agents often fail laboratory and clinical trials due to poor aqueous solubility, instability, insufficient site specificity, general toxicity, or formulation issues. Liposomes are spherical vesicles having at least one lipid bilayer. Liposomes can be used as vehicles for administration of nutrients and pharmaceutical drugs. Bioavailability and site specificity of drugs can be improved through liposome-mediated drug delivery.

### INCORPORATION BY REFERENCE

Each patent, publication, and non-patent literature cited in the application is hereby incorporated by reference in its entirety as if each was incorporated by reference individually.

### SUMMARY OF THE INVENTION

In some embodiments, the disclosure provides a lipid-based carrier comprising: a) a surface layer, wherein the surface layer comprises a prodrug, wherein the prodrug comprises a therapeutic agent covalently conjugated to a phospholipid; and b) a core, wherein the surface layer surrounds the core.

In some embodiments the disclosure provides a method of treating a condition, the method comprising administering to a subject in need thereof a therapeutically-effective amount of a lipid-based carrier, the lipid-based carrier comprising: a) a surface layer, wherein the surface layer comprises a prodrug, wherein the prodrug comprises a therapeutic agent covalently conjugated to a phospholipid; and b) a core, wherein the surface layer surrounds the core.

### BRIEF DESCRIPTION OF THE FIGURES

FIG. 1 illustrates schemes by which cytarabine can be conjugated to a phospholipid.

FIG. 2 PANEL A illustrates the synthesis of prodrugs, self-assembly of prodrug-loaded liposomes, and the use of the liposomes for treating cells in vitro. PANEL B shows the use of prodrug-loaded microbubbles and ultrasound exposure for targeted drug delivery in vitro.

FIG. 3 shows a schematic representation of treatment with microbubbles utilizing ultrasound as an extracorporeal trigger.

FIG. 4 illustrates synthetic routes used to couple cytarabine and topotecan to phospholipids.

FIG. 5 shows differential scanning calorimetry curves of 2T-P-loaded liposomes with increasing concentrations.

FIG. 6 shows differential scanning calorimetry curves of P-loaded liposomes with increasing concentrations.

FIG. 7 PANEL A shows the drug incorporation within liposomes pre- and post-extrusion of 2T-P. PANEL B shows the drug incorporation within liposomes pre- and post-extrusion of 2T-N.

FIG. 8 PANEL A shows that 2T-P-loaded liposomes remained stable throughout a period of 3 weeks. PANEL B shows that P-loaded liposomes were stable throughout a period of 3 weeks. PANEL C shows that 2T-N-loaded liposomes remained stable throughout a period of 3 weeks. PANEL D shows that N-loaded liposomes remained stable throughout a period of 3 weeks.

FIG. 9 shows the drug incorporation within liposomes pre- and post-extrusion of 2T-T.

FIG. 10 shows size distribution curves liposomes with varying amounts of 2T-T pre-extrusion (pre-ex) and post-extrusion (post-ex).

FIG. 11 shows the average diameter of liposome populations (see FIG. 10) with varying amounts of 2T-T pre- and post-extrusion

FIG. 12 shows size distribution curves liposomes with varying amounts of 2T-C pre-extrusion (_pre) and post-extrusion (_post).

FIG. 13 shows the average diameter of liposome populations (see FIG. 12) with varying amounts of 2T-C pre- and post-extrusion

FIG. 14 shows the cytotoxicity of 2T-T loaded liposomes versus the toxicity of free T (topotecan).

FIG. 15 shows the cytotoxicity of 2T-C loaded liposomes versus the toxicity of free C (cytarabine).

FIG. 16 shows in vitro ultrasound-triggered delivery of prodrug-loaded microbubbles.

FIG. 17 shows the fluorescence spectra of 2T-N-loaded liposomes at different time points with and without treatment with porcine liver esterase.

FIG. 18 shows the fluorescence spectra of empty liposomes at different time points with and without treatment with porcine liver esterase.

FIG. 19 shows the fluorescence spectra of a PBS solution at different time points with and without treatment with porcine liver esterase.

### DETAILED DESCRIPTION

Many promising natural and synthetic chemotherapy agents fail laboratory and clinical trials due to poor aqueous solubility, instability, insufficient site specificity, general toxicity, or formulation issues. Lipid based carriers such as liposomes, nanodroplets, or microbubbles can be used as vehicles for administration of nutrients and pharmaceutical drugs, and can significantly improve the bioavailability and site specificity of therapeutic agents.

Liposomes are spherical vesicles having at least one lipid bilayer. A liposome has a core (e.g., aqueous solution core) surrounded by a hydrophobic membrane in the form of a lipid bilayer. The major types of liposomes are multilamellar vesicles (MLVs) that have several lamellar phase lipid bilayers, small unilamellar liposome vesicles (SUVs) with one lipid bilayer, large unilamellar vesicles (LUVs), and cochleate vesicles. To deliver molecules (e.g., therapeutic agents) to a site of action, the lipid bilayers of liposomes can fuse with other bilayers, such as the cell membrane. Liposomes can be used as carriers of dietary or nutritional supplements, or for targeted drug delivery.

Microbubbles are small, gas-filled bubbles, typically between 0.5 μm and 10 μm in diameter. The cores of microbubbles are gaseous, which is surrounded by a shell composed of, for example, polymers, lipids, lipopolymers, proteins, surfactants, or a combination thereof.

Microbubbles are used as contrast agents in medical imaging and as carriers for targeted drug delivery. Microbubbles resonate vigorously at the high frequencies used in ultrasound scans, and reflect the strong ultrasound waves more effectively than body tissue. Microbubbles are approximately the same size as red blood cells, exhibit similar rheology in blood vessels, and can be used to measure blood flow in organs or tumors.

Nanodroplets are small, liquid filled bubbles that are smaller than microbubbles. In some embodiments, the shell of a nanodroplet comprises, for example, lipids or phospholipids.

Nanodroplets can be filled with liquids that vaporize easily. Upon vaporization of the liquid core of a nanodroplet, the nanodroplet transitions to a microbubble. Non-limiting examples of liquid cores utilized in nanodroplets include perfluorocarbon and perfluorobutane.

The disclosure describes the development and use of a set of lipid prodrugs, the molecular interactions of the lipid prodrugs within lipid-based carriers, and the efficacy of the lipid prodrugs in vitro and in vivo using ultrasound. Non-limiting examples of lipid based carriers include liposomes and microbubbles. The synthetic strategy described herein allows potent lipid prodrugs to be loaded into lipid based-based carriers such as liposomes and microbubbles to form a prodrug loaded lipid based carrier (PLLBC). In some embodiments, PLLBCs can be used as ultrasound contrast agents, and coupled with ultrasound for site-directed therapy that allows real-time visualization of the target (e.g., malignant tumors) and the arrival of the contrast agent at the target site.

Lipid prodrugs can utilize lipid-based drug carriers, targeted delivery strategies, and ultrasound-mediated techniques to achieve better performance by: 1) increasing drug payload; 2) minimizing purification and solubility issues with vehicle self-assembly; 3) preventing premature drug release from the vehicle; 4) remaining activated with ultrasound imaging and therapeutic techniques that put the drug in proximity to target cells; and 5) maintaining drug potency once rapidly cleaved intracellularly. The conjugation of a lipid and a drug via a cleavable ester bond that is sterically unhindered allows a drug to self-assemble at high concentrations into a carrier that is activated by ultrasound; and upon intracellular uptake, release a potent and fast-acting drug.

The lipid prodrugs and methods described herein can be used to target tumors, such as irressectable pancreatic, liver, and brain tumors. In some embodiments, the prodrugs and methods descried herein can be used to treat pancreatic cancer by targeting the stromal matrix of a subject. In some embodiments, the methods described herein can be used for drugs that possess toxicity or solubility issues. In some embodiments, targeting ligands can be added to the microbubble shells described herein.

### Lipid Prodrugs

In some embodiments, the disclosure describes lipid prodrugs that can first self-assemble into lipid-based carriers (e.g. liposomes, nanodroplets, or microbubbles) and then remain inactive until activated intracellularly after deposition with an external stimulus. In some embodiments, a synthetic approach described herein attaches activated phospholipids to a therapeutic agent that has a sterically-unhindered hydroxyl group attachment site. This structure allows for simple conjugation via esterification. In some embodiments, a synthetic approach described herein attaches activated phospholipids to a therapeutic agent that has a sterically-unhindered amine group attachment site. This structure allows for simple conjugation via amidation. Following conjugation of therapeutic agents to activated phospholipids, an enzymatic reaction cleavage can cause cleavage in a biological environment.

The disclosure also describes methods for inserting drugs into lipid-based carriers. Non-limiting examples of lipid based carriers include liposomes, nanodroplets, and microbubbles. In some embodiments, the disclosure utilizes FDA-accepted drugs or drugs that are well-characterized but are limited in clinical application due to solubility issues or extreme potency.

To facilitate loading into lipid based carriers, drugs can be attached to activated phospholipids to form a prodrug. Prodrugs can then be incorporated into lipid-based carriers by, for example, self-assembly to form a PLLBC. PLLBCs (e.g., liposomes, nanodroplets, or microbubbles) of the disclosure can comprise a therapeutic agent, for example, an anti-viral agent, anti-bacterial agent, anti-cancer agent, a neurotransmitter, a protein, dermatological agents, cosmetic agents, chelating agents, or a biologic. In some embodiments, PLLBCs of the disclosure comprise combinations of prodrugs. In some embodiments, a PLLBC of the disclosure comprise a dye molecule.

A PLLBC of the disclosure, such as a liposome, nanodroplet, or microbubble can surround a core material. Non-limiting examples of core materials include gases such as sulfurhexafluoride (SF₆) or perfluoropropane; solids such as titanium nitride, super paramagnetic iron oxide, gold, silver, iron, copper, zinc, titanium, platinum, gadolinium, and palladium; semiconductors such as silica; inorganic materials; organic materials; aqueous solutions; and liquids such as perfluorocarbon and perfluorobutane.

A PLLBC, of the disclosure can comprise an antiviral agent. The antiviral agents can minimize symptoms and infectivity, and shorten the duration of illness. A PLLBC of the disclosure can comprise antiviral agents that inhibit the attachment and penetration of a virus to the host cell, release of nuclei acid, replication of the viral genome, translation of viral mRNA, assembly of viral components, or release of new viruses from the host cell. In some embodiments, the PLLBC of the disclosure can comprise an antiviral agent, for example, amantadine, a nucleoside analogue (e.g., acyclovir, ganciclovir, foscarnet), a nucleoside reverse transcriptase inhibitor (NRTI; e.g., lamivudine), a non-nucleoside reverse transcriptase inhibitor (e.g., nevirapine, efavirenz), interferon alpha, a protease inhibitor (e.g., boceprevir), or a neuraminidase inhibitor (e.g., oseltamivir).

In some embodiments, a PLLBC, of the disclosure can comprise an anti-viral agent against influenza viruses, such as an ion channel blocker (e.g., amantadine, rimantadine) or a neuraminidase inhibitor (e.g., oseltamivir or zanamivir). In some embodiments, a PLLBC of the disclosure can comprise an anti-viral agent against herpes viruses, such as guanosine analogues (e.g., acyclovir, penciclovir, valacyclovir, famciclovir, ganciclovir, valganciclovir), or a direct viral DNA polymerase inhibitor (e.g., foscarnet, cidofovir). In some embodiments, a PLLBC of the disclosure can comprise an anti-viral agent against hepatitis B and C, such as a nucleotide analogue (e.g., tenofovir, adefovir, lamivudine, entecavir, telbivudine), an anti-viral and immunomodulatory agent via intercellular and intracellular mechanisms (e.g., PEG-interferon-alpha), guanosine analogues (e.g., ribavarin), protease inhibitors (e.g., simeprevir), non-nucleoside polymerase (NS5A) inhibitors (e.g., ledipasvir, velpatasvir), or non-nucleoside polymerase (NS5B) inhibitors (e.g., sofosbuvir).

A PLLBC of the disclosure can comprise an antibacterial agent such as anilides, quinolones, sulfonamides, penicillins, protein synthesis inhibitors (e.g. macrolides, lincosamides, tetracyclines), biguanides, bisphenols, halophenols, phenols, cresols, or quaternary ammonium compounds. In some embodiments, a PLLBC of the disclosure can comprise triclocarban, chlorhexidine, alexidine, polymeric biguanides, hexachlorophene, p-chloro-m-xylenol (PCMX), phenol, cresol, cetrimide, benzalkonium chloride, norflaxacin, polymyxin B, oxacillin, dicloxaccilin, tetracycline, vancomycin, penicillin, rifamycin, lipiarmycind, streptomycin, amphotericin B, cephalosporin or cetylpyridinium chloride.

A PLLBC of the disclosure can comprise an anticancer agent. Non-limiting examples of anti-cancer agents include a polyfunctional alkylating agent, alkylating agent, purine antagonist, pyrimidine antagonist, plant alkaloids, antibiotics, hormonal agents, or other anticancer drugs. In some embodiments, a PLLBC of the disclosure can comprise an anticancer agent such as podophyllotoxin (P), 7-(3,5-Dibromophenyl)-2-hydroxy-7,11-dihydrobenzo[h]-furo[3,4-b]quinolin-8(10H)-one (N), cyclophosphamide, fosfamide, mechloroethamine, melphalan (Alkeran®), chlorambucil (Leukeran™), thiopeta (Thioplex®), busulfan (Myleran®), carmustine, lomustine, semustine, procarbazine (Matulane®), dacarbazine (DTIC), altretamine (Hexalen®), cisplatin (Platinol®), methotrexate, mercaptopurine (6-MP), thioguanine (6-TG), fludarabine phosphate, cladribine (Leustatin®), pentostatin (Nipent®), fluorouracil (5-FU), cytarabine (Ara-C), azacitidine, vinblastine (Velban®), vincristine (Oncovin®), etoposide (VP-16, VePe-sid®), teniposide (Vumon®), topotecan (Hycamtin®), irinotecan (Camptosar®), paclitaxel (Taxol®), docetaxel (Taxotere®), anthracyclines (e.g., doxorubicin, daunorubicin), dactinomycin (Cosmegen®), idarubincin (Idamycin®), plicamycin (Mithramycin®), mitomycin (Mutamycin®), bleomycin (Blenoxane®), tamoxifen (Nolvadex®), flutamide (Eulexin®), gonadotropin-releasing hormone agonists (e.g., leuprolide, goserelin), aromatase inhibitors (e.g., aminoglutethimide, anastrozole (Arimidex®), amsacrine, gemcitabine, melphelan, methotrexate, hydroxyurea (Hydrea®), asparaginase (El-spar®), mitoxantrone (Novantrone®), mitotane, retinoic acid derivatives, bone marrow growth factors, or aminfostine.

A PLLBC of the disclosure can comprise a neurotransmitter. Non-limiting examples of neurotransmitters include amino acids, gasotransmitters, monoamines, trace amines, peptides, purines, or other neurotransmitters. In some embodiments, the liposomes, nanodroplets, or microbubbles of the disclosure can comprise a neurotransmitter, for example, glutamate, aspartate, D-serine, γ-aminobutyric acid (GABA), glycine, dopamine, norepinephrine, epinephrine, histamine, serotonin, phenethylamine, N-methylphenethylamine, tyramine, 3-iodothyronamine, octopamine, tryptamine, oxytocin, somatostatin, substance P, cocaine and amphetamine regulated transcript, opioid peptides, adenosine triphosphate (ATP), adenosine, acetylcholine (ACh), and anandamide.

A PLLBC of the disclosure can comprise a protein or biologic. Non-limiting examples of proteins or biologics include peptides; peptide fragments; antibodies such as divalent antibodies, monovalent antibodies, polyclonal antibodies, and monoclonal antibodies; antibody fragments; and nanobodies.

### Method of Synthesis

The present disclosure describes methods of synthesizing PLLBCs such as liposomes, nanodroplets, or microbubbles. A prodrug-loaded liposomes or microbubble can comprise a therapeutic agent in the respective lipid layers. A prodrug-loaded liposome of the disclosure can comprise a therapeutic agent in the lipid bilayers of the liposome, and a prodrug-loaded microbubble of the disclosure can comprise a therapeutic agent in the lipid monolayers of the microbubbles.

In some embodiments, a PLLBC can be produced by incorporating lipid prodrugs into the lipid-shell of a lipid-based carrier. In some embodiments, incorporating lipid prodrugs into the lipid-shell of a lipid-based carrier can eliminate leaking, covalently bind the drug to the lipid-based carrier, and deliver a dual-targeting strategy in one dose. In some embodiments, incorporating lipid prodrugs into the lipid-shell of a lipid-based carrier can impact the pharmacokinetics (PK) or pharmacodynamics (PD) of a drug.

In some embodiments, self-assembly is used to incorporate a prodrug into the lipid-shell of a lipid-based carrier. In some embodiments, incorporating a prodrug into the lipid-shell of a lipid-based carrier can minimize or eliminate purification steps prior to administration of the prodrug into a subject. In some embodiments, prodrug-loaded microbubbles can be separated from prodrug-loaded liposomes prior to administration using centrifugation. In some embodiments, incorporating a prodrug into the lipid-shell of a lipid-based carrier can increase the amount of a drug that is delivered to a site of interest within a subject while minimizing the systemic dose to the subject.

The PLLBCs (e.g. liposomes, nanodroplets, or microbubbles) of the disclosure can be assembled using solutions prodrugs produced by the conjugation of drug molecules or therapeutic agents to phospholipids. The drug molecule or therapeutic agent can be conjugated to an activated phospholipid, for example, a maleimido (MAL) phospholipid, activated carboxylic acid (NHS)-phospholipid, glutaryl (Glu)-phospholipid, 7-nitrobenz-2-oxa-1,3-diazol-4-yl (NBD)-phospholipid, or dithiopyridinl (PDP)-Phospholipid. In some embodiments, the drug molecule is conjugated to a MAL phospholipid, such as N-(3-maleimide-1-oxopropyl)-L-α-phosphatidylethanolamine, distearoyl (DSPE-MAL); N-(3-maleimide-1-oxopropyl)-L-α-phosphatidylethanolamine, dimyristoyl (DMPE-MAL); N-(3-maleimide−1-oxopropyl)-L-α-phosphatidylethanolamine, 1-palmitoyl-2-oleoyl (POPE-MAL); or N-(3-maleimide-1-oxopropyl)-L-α-phosphatidylethanolamine, dipalmitoyl (DPPE-MAL). In some embodiments, the drug molecule is conjugated to an NHS-phospholipid, such as N-(succinimidyloxy-glutaryl)-L-α-phosphatidylethanolamine, distearoyl (DSPE-NHS); N-(succinimidyloxy-glutarvl)-L-α-phosphatidylethanolamine, dioleoyl (DOPE-NHS); N-(succinimidyloxy-glutaryl)-L-α-phosphatidylethanolamine, 1-palmitoyl-2-oleoyl (POPE-NHS); N-(succinimidyloxy-glutaryl)-L-α-phosphatidylethanolamine, dipalmitoyl (DPPE-NHS); or N-(succinimidyloxy-glutaryl)-L-α-phosphatidylethanolamine, dimyristoyl (DMPE-NHS).

In some embodiments, the drug molecule is conjugated to a Glu-phospholipid, such as N-glutaryl-L-α-phosphatidylethanolamine, distearoyl (DSPE-Glu); N-glutaryl-L-α-phosphatidylethanolamine, dipalmitoyl (DPPE-Glu); N-glutaryl-L-α-phosphatidylethanolamine, dimyristoyl (DMPE-Glu); N-glutaryl-L-α-phosphatidylethanolamine, dioleoyl (DOPE-Glu); or N-glutaryl-L-α-phosphatidylethanolamine, 1-palmitoyl-2-oleoyl (POPE-Glu). In some embodiments, the drug molecule is conjugated to a PDP-phospholipid, such as N-[3-(2-pyridinyldithio)-1-oxopropyl]-L-α-phosphatidylethanolamine, dipalmitoyl (DPPE-PDP).

SCHEME 1 shows a non-limiting example of a synthetic scheme that can be used to produce prodrugs by synthesizing drug-conjugated phospholipids. In some embodiments, prodrugs are used in assembling PLLBCs of the disclosure such as liposomes, nanodroplets, or microbubbles. To produce a prodrug, a drug molecule or therapeutic agent comprising a hydroxyl group, amino (amido) group, carboxylic group, or mercapto group is conjugated to an activated phospholipid in the presence of a dehydrating agent and nucleophilic catalyst. In some embodiments, the activated phospholipid is a Glu-phospholipid, for example, DPPE-Glu. In some embodiments, the dehydrating agent is N,N′-dicylohexylcarbodiimide (DCC), and the nucleophilic catalyst is 4-dimethylaminopyridine (DMAP).

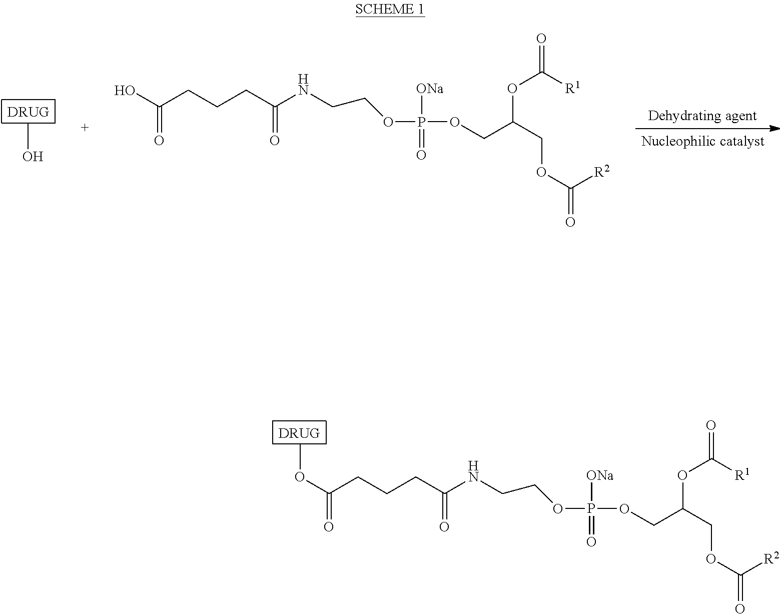

The lipid prodrugs can have hydrophobic tails comprising saturated fatty acids or unsaturated fatty acids. In some embodiments prodrugs are double tailed. In some embodiments, the prodrug molecules have hydrophobic tails comprising saturated fatty acids that are straight chain alkylene moieties. In some embodiments, the prodrug molecules have hydrophobic tails comprising unsaturated fatty acids that are straight chain alkenylene moieties. In some embodiments, each R¹ and R² is —(CH₂)ₙ—, wherein n is from about 5 to about 24. In some embodiments, each R¹ and R² is —(CH₂)ₙ—, wherein n is 10. In some embodiments, each R¹ and R² is —(CH₂)ₙ—, wherein n is 12. In some embodiments, each R¹ and R² is —(CH₂)ₙ—, wherein n is 20.

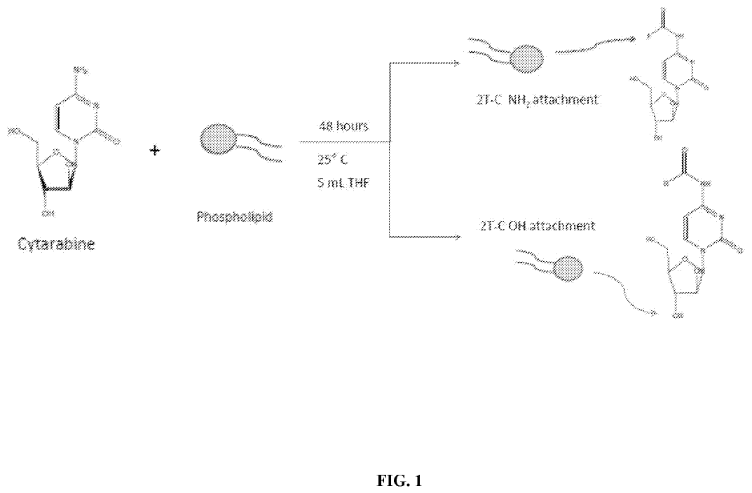

Additional synthetic schemes can also be used to produce prodrugs by synthesizing drug-conjugated phospholipids. In some embodiments, a therapeutic agent such as cytarabine can be conjugated to a phospholipid through either an amide bond or an ester bond as shown in FIG. 1.

A prodrug solution of the disclosure can be used to assemble prodrug-loaded liposomes. Prodrug-loaded liposomes can be assembled by mixing and sonicating a solution of the drug-conjugated phospholipid (i.e. prodrug) with a phospholipid solution and a PEGylated phospholipid solution.

A prodrug solution of the disclosure can be used to assemble prodrug-loaded microbubbles. Prodrug-loaded microbubbles can be assembled by mixing and sonicating a solution of the drug-conjugated phospholipid (i.e. prodrug) with a phospholipid solution and PEGylated phospholipid solution, and purging the resulting solution with a gas to form the gaseous core of the microbubbles.

In some embodiments, a PEGylated phospholipid solution used to assemble drug loaded lipid carriers such as drug-loaded liposomes or drug-loaded microbubbles comprises for example, DSPE-PEG (1,2-distearoyl-sn-glycero-3-phosphoethanolamine-N-(methoxy (polyethyleneglycol) ammonium salt), DPPE-PEG ((N-(Methylpolyoxyethylene oxycarbonyl)-1,2-dipalmitoyl-sn-glycero-3-phosphoethanolamine-N-(methoxy (polyethyleneglycol) ammonium salt), DMPE-PEG (N-(Methylpolyoxyethylene oxycarbonyl)-1,2-dimyristoyl-sn-glycero-3-phosphoethanolamine-N-(methoxy (polyethyleneglycol) ammonium salt), or any combination thereof. In some embodiments, a PEGylated phospholipid solution use to assemble drug loaded lipid carriers such as drug-loaded liposomes or drug-loaded microbubbles comprises for example, DSPE-PEG2000 (1,2-distearoyl-sn-glycero-3-phosphoethanolamine-N-(methoxy (polyethyleneglycol) 2000) ammonium salt), DSPE-PEG5000 (1,2-distearoyl-sn-glycero-3-phosphoethanolamine-N-(methoxy (polyethyleneglycol) 5000) ammonium salt), DSPE-PEG2000 carboxylic acid (1,2-distearoyl-sn-glycero-3-phosphoethanolamine-N-[carboxy(polyethylene glycol)-2000] (sodium salt)), DSPE-PEG5000 DBCO (1,2-distearoyl-sn-glycero-3-phosphoethanolamine-n-[dibenzocyclooctyl(polyethylene glycol)-5000] (ammonium salt)), DSPE-PEG5000 amine (1,2-distearoyl-sn-glycero-3-phosphoethanolamine-N-[amino(polyethylene glycol)-5000] (ammonium salt)), DSPE-PEG5000 maleimide (1,2-distearoyl-sn-glycero-3-phosphoethanolamine-N-[maleimide(polyethylene glycol)-5000](ammonium salt)), DSPE-PEG2000-TMS (1,2-distearoyl-sn-glycero-3-phosphoethanolamine-N-[10-(trimethoxysilyl)undecanamide(polyethylene glycol)-2000] (triethylammonium salt)), DSPE-PEG5000 azide (1,2-distearoyl-sn-glycero-3-phosphoethanolamine-N-[azido(polyethylene glycol)-5000] (ammonium salt)), DSPE-PEG2000-square (1,2-distearoyl-sn-glycero-3-phosphoethanolamine-N-[square(polyethylene glycol)-2000] (sodium salt)), DSPE-PEG2000-DBCO (1,2-distearoyl-sn-glycero-3-phosphoethanolamine-N-[dibenzocyclooctyl(polyethylene glycol)-2000] (ammonium salt)), DSPE-PEG2000 azide (1,2-distearoyl-sn-glycero-3-phosphoethanolamine-N-[azido(polyethylene glycol)-2000] (ammonium salt)), DSPE-PEG2000 biotin (1,2-distearoyl-sn-glycero-3-phosphoethanolamine-N-[biotinyl(polyethylene glycol)-2000] (ammonium salt)), DSPE-PEG2000 amine (1,2-distearoyl-sn-glycero-3-phosphoethanolamine-N-[amino(polyethylene glycol)-2000] (ammonium salt)), DSPE-PEG2000 PDP (1,2-distearoyl-sn-glycero-3-phosphoethanolamine-N-[PDP(polyethylene glycol)-2000] (ammonium salt)), DSPE-PEG2000 maleimide (1,2-distearoyl-sn-glycero-3-phosphoethanolamine-N-[maleimide(polyethylene glycol)-2000] (ammonium salt)), DSPE-PEG2000 folate (1,2-distearoyl-sn-glycero-3-phosphoethanolamine-N-[folate(polyethylene glycol)-2000] (ammonium salt)), DSPE-PEG5000 folate (1,2-distearoyl-sn-glycero-3-phosphoethanolamine-N-[folate(polyethylene glycol)-5000] (ammonium salt)), DSPE-PEG2000 cyanur (1,2-distearoyl-sn-glycero-3-phosphoethanolamine-N-[cyanur(polyethylene glycol)-2000] (ammonium salt)), DSPE-PEG2000 succinyl (1,2-distearoyl-sn-glycero-3-phosphoethanolamine-N-[succinyl(polyethylene glycol)-2000] (ammonium salt)), DSPE-PEG2000-N-Cyanine 5, DSPE-PEG2000-N-Cyanine 7, Bis-DSPE-PEG2000 (1,2-dipalmitoyl-sn-glycero-3-phosphoethanolamine-N-[azido(polyethylene glycol)-2000 (ammonium salt), DMPE-PEG200 (N-(Methylpolyoxyethylene oxycarbonyl)-1,2-dimyristoyl-sn-glycero-3-phosphoethanolamine-N-(methoxy (polyethyleneglycol) 2000) ammonium salt)), DMPE-PEG5000 (N-(Methylpolyoxyethylene oxycarbonyl)-1,2-dimyristoyl-sn-glycero-3-phosphoethanolamine-N-(methoxy (polyethyleneglycol) 5000) ammonium salt)), DPPE-PEG2000 (N-(Methylpolyoxyethylene oxycarbonyl)-1,2-dipalmitoyl-sn-glycero-3-phosphoethanolamine-N-(methoxy (polyethyleneglycol) 2000) ammonium salt)), DPPE-PEG5000 (N-(Methylpolyoxyethylene oxycarbonyl)-1,2-dipalmitoyl-sn-glycero-3-phosphoethanolamine-N-(methoxy (polyethyleneglycol) 5000) ammonium salt)), methoxy-PEG lipids such as 1,2-dipalmitoyl-sn-glycero-3-phosphoethanolamine-N-[methoxy(polyethylene glycol)] (ammonium salt), 1,2-dimyristoyl-sn-glycero-3-phosphoethanolamine-N-[methoxy(polyethylene glycol)] (ammonium salt), 1,2-distearoyl-sn-glycero-3-phosphoethanolamine-N-[methoxy(polyethylene glycol)] (ammonium salt), 1,2-dioleoyl-sn-glycero-3-phosphoethanolamine-N-[methoxy(polyethylene glycol)] (ammonium salt) with methoxy-PEG weights of 350, 550, 750, 1000, 2000, 3000, or 5000 daltons; or any combination thereof.

In some embodiments a phospholipid solution of the disclosure that can be used to assemble drug-loaded lipid based carriers such as liposomes, nanodroplets, or microbubbles comprises a natural phospholipid derivative, for example, egg phosphatidylcholine (PC), egg phosphatidylglycerol (PG), soy PC, hydrogenated soy PC, or sphingomyelin. In some embodiments, a phospholipid solution used to assemble drug-loaded lipid based carriers such as liposomes, nanodroplets, or microbubbles of the disclosure is a synthetic phospholipid derivative, for example, phosphatidic acid (e.g., 1,2-Dimyristoyl-sn-glycero-3-phosphate (DMPA), 1,2-Dipalmitoyl-sn-glycero-3-phosphate (DPPA), 1,2-Distearoyl-sn-glycero-3-phosphate (DSPA)), phosphatidylcholine (e.g., 1,2-Didecanoyl-snr-glycero-3-phosphocholine (DDPC), 1,2-Dilauroyl-sn-glycero-3-phosphocholine (DLPC), 1,2-Dimyristoyl-sn-glycero-3-phosphocholine (DMPC), 1,2-Dipalmitoyl-sn-glycero-3-phosphocholine (DPPC), 1,2-Distearoyl-sn-glycero-3-phosphocholine (DSPC), 1,2-Dioleoyl-sn-glycero-3-phosphocholine (DOPC), 1-Palmitoyl-2-oleoyl-sn-glycero-3-phosphocholine (POPC), 1,2-Dierucoyl-sn-glycero-3-phosphocholine (DEPC)), phosphatidylglycerol (e.g., 1,2-Dimyristoyl-sn-glycero-3-phospho-rac-(1-glycerol) (DMPG), 1,2-Dipalmitoyl-sn-glycero-3-phospho-rac-(1-glycerol) (DPPG), 1,2-Distearoyl-sn-glycero-3-phospho-rac-(1-glycerol) (DSPG), 2-Oleoyl-1-palmitoyl-sn-glycero-3-phospho-rac-(1-glycerol) (POPG)), phosphatidylethanolamine (e.g., 1,2-Dimyristoyl-sn-glycero-3-phosphoethanolamine (DMPE), 1,2-Dipalmitoyl-sn-glycero-3-phosphoethanolamine (DPPE), 1,2-Distearoyl-sn-glycero-3-phosphoethanolamine (DSPE), 1,2-Dioleoyl-snr-glycero-3-phosphoethanolamine (DOPE)), phosphatidylserine (e.g., 1,2-Dioleoyl-sn-glycero-3-phosphoserine (DOPS)), 1-Palmitoyl-2-oleoyl-sn-glycero-3-phosphoethanolamine (POPE), dipalmitoylphosphatidylcholine (DPPtdCho), 1-stearoyl-2-[(E)-4-(4-((4-butylphenyl)diazenyl)phenyl)butanoyl]-sn-glycero-3-phosphocholine, 1-stearoyl-2-[(E)-4-(4-((4-butylphenyl)diazenyl)phenyl)butanoyl]-sn-glycerol, 1-stearoyl-2-[(E)-4-(4-((4-butylphenyl)diazenyl)phenyl)butanoyl]-sn-glycero-3-phosphocholine, N-[(E)-4-(4-((4-butylphenyl)diazenyl)phenyl)butanoyl]-D-erythro-sphingosylphosphorylcholine, (E)-4-(4-((4-butylphenyl)diazenyl)phenyl)-N-(3-hydroxy-4-methoxybenzyl)butanamide, 4-Butyl-Azo-4:0-Acid-1, 1,2-dipalmitoyl-sn-glycero-3-phosphoethanolamine-N-diethylenetriaminepentaacetic acid (gadolinium salt), 1,2-distearoyl-sn-glycero-3-phosphoethanolamine-N-diethylenetriaminepentaacetic acid (gadolinium salt), bis(1,2-dimyristoyl-sn-glycero-3-phosphoethanolamine)-N—N′-diethylenetriaminepentaacetic acid (gadolinium salt), bis(1,2-dipalmitoyl-sn-glycero-3-phosphoethanolamine)-N—N′-diethylenetriaminepentaacetic acid (gadolinium salt), bis(1,2-distearoyl-sn-glycero-3-phosphoethanolamine)-N—N′-diethylenetriaminepentaacetic acid (gadolinium salt), a polyglycerin-phospholipid, a functionalized phospholipid, or a terminal-activated phospholipid or pharmaceutically-acceptable salt thereof. In some embodiments, a phospholipid solution of the disclosure that can be used to assemble drug-loaded lipid based carriers such as liposomes, nanodroplets, or microbubbles comprises a combination of any of the above phospholipids.

### Characteristics of Lipid-Based Carrier Formulations

Size: In some embodiments, a lipid-based carrier (e.g. a liposome, nanodroplet, or microbubble) of the present disclosure can have a diameter of about 70 nm to about 10 μm. In some embodiments, a lipid-based carrier of the present disclosure can have a diameter of about 70 nm to about 100 nm, about 70 nm to about 150 nm, about 70 nm to about 200 nm, about 70 nm to about 250 nm, about 70 nm to about 300 nm, about 70 nm to about 350 nm, about 70 nm to about 400 nm, about 70 nm to about 450 nm, about 70 nm to about 500 nm, about 70 nm to about 550 nm, about 70 nm to about 600 nm, about 70 nm to about 900 nm, about 70 nm to about 1 μm, about 70 nm to about 5 μm, about 70 nm to about 10 μm, about 100 nm to about 150 nm, about 100 nm to about 200 nm, about 100 nm to about 250 nm, about 100 nm to about 300 nm, about 100 nm to about 350 nm, about 100 nm to about 400 nm, about 100 nm to about 450 nm, about 100 nm to about 500 nm, about 100 nm to about 550 nm, about 100 nm to about 600 nm, about 100 nm to about 900 nm, about 100 nm to about 1 μm, about 100 nm to about 5 μm, about 100 nm to about 10 μm, about 150 nm to about 200 nm, about 150 nm to about 250 nm, about 150 nm to about 300 nm, about 150 nm to about 350 nm, about 150 nm to about 400 nm, about 150 nm to about 450 nm, about 150 nm to about 500 nm, about 150 nm to about 550 nm, about 150 nm to about 600 nm, about 150 nm to about 900 nm, about 150 nm to about 1 μm, about 150 nm to about 5 μm, about 150 nm to about 10 μm, about 200 nm to about 250 nm, about 200 nm to about 300 nm, about 200 nm to about 350 nm, about 200 nm to about 400 nm, about 200 nm to about 450 nm, about 200 nm to about 500 nm, about 200 nm to about 550 nm, about 200 nm to about 600 nm, about 200 nm to about 900 nm, about 200 nm to about 1 μm, about 200 nm to about 5 μm, about 200 to about 10 μm, about 250 nm to about 300 nm, about 250 nm to about 350 nm, about 250 nm to about 400 nm, about 250 nm to about 450 nm, about 250 nm to about 500 nm, about 250 nm to about 550 nm, about 250 nm to about 600 nm, about 250 nm to about 900 nm, about 250 nm to about 1 μm, about 250 nm to about 5 μm, about 250 nm to about 10 μm, about 300 nm to about 350 nm, about 300 nm to about 400 nm, about 300 nm to about 450 nm, about 300 nm to about 500 nm, about 300 nm to about 550 nm, about 300 nm to about 600 nm, about 300 nm to about 900 nm, about 300 nm to about 1 μm, about 300 nm to about 5 μm, about 300 nm to about 10 μm, about 350 nm to about 400 nm, about 350 nm to about 450 nm, about 350 nm to about 500 nm, about 350 nm to about 550 nm, about 350 nm to about 600 nm, about 350 nm to about 900 nm, about 350 nm to about 1 μm about 350 nm to about 5 μm, about 350 nm to about 10 μm, about 400 nm to about 450 nm, about 400 nm to about 500 nm, about 400 nm to about 550 nm, about 400 nm to about 600 nm, about 400 nm to about 900 nm, about 400 nm to about 1 μm, about 400 nm to about 5 μm, about 400 nm to about 10 μm, about 450 nm to about 500 nm, about 450 nm to about 550 nm, about 450 nm to about 600 nm, about 450 nm to about 900 nm, about 450 nm to about 1 μm, about 450 nm to about 5 μm, about 450 nm to about 10 μm, about 500 nm to about 550 nm, about 500 nm to about 600 nm, about 500 nm to about 900 nm, about 500 nm to about 1 μm, about 500 nm to about 5 μm, about 500 nm to about 10 μm, about 550 nm to about 600 nm, about 550 nm to about 900 nm, about 550 nm to about 1 μm, about 550 nm to about 5 μm, about 550 nm to about 10 μm, about 600 nm to about 900 nm, about 600 nm to about 1 μm, about 600 nm to about 5 μm, about 600 nm to about 10 μm, about 900 nm to about 1 μm, about 900 nm to about 5 μm, about 900 nm to about 10 μm, about 1 μm to about 5 μm, about 1 μm to about 10 μm, or about 5 μm to about 10 μm. In some embodiments, a lipid-based carrier of the present disclosure can have a diameter of about 70 nm, about 100 nm, about 150 nm, about 200 nm, about 250 nm, about 300 nm, about 350 nm, about 400 nm, about 450 nm, about 500 nm, about 550 nm, about 600 nm, about 900 nm, about 1 μm, about 5 μm, or about 10 μm. In some embodiments, a lipid-based carrier of the present disclosure can have a diameter of at least about 70 nm, about 100 nm, about 150 nm, about 200 nm, about 250 nm, about 300 nm, about 350 nm, about 400 nm, about 450 nm, about 500 nm, about 550 nm, about 600 nm, about 900 nm, about 1 μm, or about 5 μm. In some embodiments, a lipid-based carrier of the present disclosure can have a diameter of at most about 100 nm, about 150 nm, about 200 nm, about 250 nm, about 300 nm, about 350 nm, about 400 nm, about 450 nm, about 500 nm, about 550 nm, about 600 nm, about 900 nm, about 1 μm, about 5 μm, or about 10 μm.

A lipid-based carrier (e.g. a liposome, nanodroplet, or microbubble) of the present disclosure can be part of a formulation. In some embodiments, formulations of the present disclosure can contain lipid-based carriers with a mean particle diameter of about 70 nm to about 10 μm. In some embodiments, formulations of the present disclosure can contain lipid-based carriers with a mean particle diameter of about 70 nm to about 100 nm, about 70 nm to about 150 nm, about 70 nm to about 200 nm, about 70 nm to about 250 nm, about 70 nm to about 300 nm, about 70 nm to about 350 nm, about 70 nm to about 400 nm, about 70 nm to about 450 nm, about 70 nm to about 500 nm, about 70 nm to about 550 nm, about 70 nm to about 600 nm, about 70 nm to about 900 nm, about 70 nm to about 1 μm, about 70 nm to about 5 μm, about 70 nm to about 10 μm, about 100 nm to about 150 nm, about 100 nm to about 200 nm, about 100 nm to about 250 nm, about 100 nm to about 300 nm, about 100 nm to about 350 nm, about 100 nm to about 400 nm, about 100 nm to about 450 nm, about 100 nm to about 500 nm, about 100 nm to about 550 nm, about 100 nm to about 600 nm, about 100 nm to about 900 nm, about 100 nm to about 1 μm, about 100 nm to about 5 μm, about 100 nm to about 10 μm, about 150 nm to about 200 nm, about 150 nm to about 250 nm, about 150 nm to about 300 nm, about 150 nm to about 350 nm, about 150 nm to about 400 nm, about 150 nm to about 450 nm, about 150 nm to about 500 nm, about 150 nm to about 550 nm, about 150 nm to about 600 nm, about 150 nm to about 900 nm, about 150 nm to about 1 μm, about 150 nm to about 5 μm, about 150 nm to about 10 μm, about 200 nm to about 250 nm, about 200 nm to about 300 nm, about 200 nm to about 350 nm, about 200 nm to about 400 nm, about 200 nm to about 450 nm, about 200 nm to about 500 nm, about 200 nm to about 550 nm, about 200 nm to about 600 nm, about 200 nm to about 900 nm, about 200 nm to about 1 μm, about 200 nm to about 5 μm, about 200 to about 10 μm, about 250 nm to about 300 nm, about 250 nm to about 350 nm, about 250 nm to about 400 nm, about 250 nm to about 450 nm, about 250 nm to about 500 nm, about 250 nm to about 550 nm, about 250 nm to about 600 nm, about 250 nm to about 900 nm, about 250 nm to about 1 μm, about 250 nm to about 5 μm, about 250 nm to about 10 μm, about 300 nm to about 350 nm, about 300 nm to about 400 nm, about 300 nm to about 450 nm, about 300 nm to about 500 nm, about 300 nm to about 550 nm, about 300 nm to about 600 nm, about 300 nm to about 900 nm, about 300 nm to about 1 μm, about 300 nm to about 5 μm, about 300 nm to about 10 μm, about 350 nm to about 400 nm, about 350 nm to about 450 nm, about 350 nm to about 500 nm, about 350 nm to about 550 nm, about 350 nm to about 600 nm, about 350 nm to about 900 nm, about 350 nm to about 1 μmm about 350 nm to about 5 μm, about 350 nm to about 10 μm, about 400 nm to about 450 nm, about 400 nm to about 500 nm, about 400 nm to about 550 nm, about 400 nm to about 600 nm, about 400 nm to about 900 nm, about 400 nm to about 1 μm, about 400 nm to about 5 μm, about 400 nm to about 10 μm, about 450 nm to about 500 nm, about 450 nm to about 550 nm, about 450 nm to about 600 nm, about 450 nm to about 900 nm, about 450 nm to about 1 μm, about 450 nm to about 5 μm, about 450 nm to about 10 μm, about 500 nm to about 550 nm, about 500 nm to about 600 nm, about 500 nm to about 900 nm, about 500 nm to about 1 μm, about 500 nm to about 5 μm, about 500 nm to about 10 μm, about 550 nm to about 600 nm, about 550 nm to about 900 nm, about 550 nm to about 1 μm, about 550 nm to about 5 μm, about 550 nm to about 10 μm, about 600 nm to about 900 nm, about 600 nm to about 1 μm, about 600 nm to about 5 μm, about 600 nm to about 10 μm, about 900 nm to about 1 μm, about 900 nm to about 5 μm, about 900 nm to about 10 μm, about 1 μm to about 5 μm, about 1 μm to about 10 μm, or about 5 μm to about 10 μm. In some embodiments, formulations of the present disclosure can contain lipid-based carriers with a mean particle diameter of about 70 nm, about 100 nm, about 150 nm, about 200 nm, about 250 nm, about 300 nm, about 350 nm, about 400 nm, about 450 nm, about 500 nm, about 550 nm, about 600 nm, about 900 nm, about 1 μm, about 5 μm, or about 10 μm. In some embodiments, formulations of the present disclosure can contain lipid-based carriers with a mean particle diameter of at least about 70 nm, about 100 nm, about 150 nm, about 200 nm, about 250 nm, about 300 nm, about 350 nm, about 400 nm, about 450 nm, about 500 nm, about 550 nm, about 600 nm, about 900 nm, about 1 μm, or about 5 μm.

In some embodiments, formulations of the present disclosure can contain lipid-based carriers with a mean particle diameter of at most about 100 nm, about 150 nm, about 200 nm, about 250 nm, about 300 nm, about 350 nm, about 400 nm, about 450 nm, about 500 nm, about 550 nm, about 600 nm, about 900 nm, about 1 μm, about 5 μm, or about 10 μm.

Loading Capacity: The loading capacity of a lipid based carrier (e.g. a liposome, nanodroplet, or microbubble) is the amount of therapeutic agent (e.g. a prodrug) that is loaded per unit weight of the lipid-based carrier. In some embodiments, a PLLBC of the disclosure has a loading capacity of about 0% to about 99%. In some embodiments, a PLLBC of the disclosure has a loading capacity of about 0% to about 5%, about 0% to about 10%, about 0% to about 15%, about 0% to about 200%, about 0% to about 30%, about 0% to about 40%, about 0% to about 50%, about 0% to about 75%, about 0% to about 90%, about 0% to about 95%, about 0% to about 99%, about 5% to about 10%, about 5% to about 15%, about 5% to about 20%, about 5% to about 30%, about 5% to about 40%, about 5% to about 50%, about 5% to about 75%, about 5% to about 90%, about 5% to about 95%, about 5% to about 99%, about 10% to about 15%, about 10% to about 20%, about 10% to about 30%, about 10% to about 40%, about 10% to about 50%, about 10% to about 75%, about 10% to about 90%, about 10% to about 95%, about 10% to about 99%, about 15% to about 20%, about 15% to about 30%, about 15% to about 40%, about 15% to about 50%, about 15% to about 75%, about 15% to about 90%, about 15% to about 95%, about 15% to about 99%, about 20% to about 30%, about 20% to about 40%, about 20% to about 50%, about 20% to about 75%, about 20% to about 90%, about 20% to about 95%, about 20% to about 99%, about 30% to about 40%, about 30% to about 50%, about 30% to about 75%, about 30% to about 90%, about 30% to about 95%, about 30% to about 99%, about 40% to about 50%, about 40% to about 75%, about 40% to about 90%, about 40% to about 95%, about 40% to about 99%, about 50% to about 75%, about 50% to about 90%, about 50% to about 95%, about 50% to about 99%, about 75% to about 90%, about 75% to about 95%, about 75% to about 99%, about 90% to about 95%, about 90%, to about 99%, or about 95% to about 99%. In some embodiments, a PLLBC of the disclosure has a loading capacity of about 0%, about 5%, about 10%, about 15%, about 20%, about 30%, about 40%, about 50%, about 75%, about 90%, about 95%, or about 99%. In some embodiments, a PLLBC of the disclosure has a loading capacity of at least about 0%, about 5%, about 10%, about 15%, about 20%, about 30%, about 40%, about 50%, about 75%, about 90%, or about 95%. In some embodiments, a PLLBC of the disclosure has a loading capacity of at most about 5%, about 10%, about 15%, about 20%, about 30%, about 40%, about 50%, about 75%, about 90%, about 95%, or about 99%.

The loading capacity of a formulation of a lipid based carrier (e.g. a liposome, nanodroplet, or microbubble) is the amount of therapeutic agent (e.g. a prodrug) that is loaded into a lipid-based carrier of the formulation per unit weight of the lipid-based carrier in the formulation. In some embodiments, a formulation of a PLLBC of the disclosure has a loading capacity of about 0% to about 99%. In some embodiments, a formulation of a PLLBC of the disclosure has a loading capacity of about 0% to about 5%, about 0% to about 10%, about 0% to about 15%, about 0% to about 20%, about 0% to about 30%, about 0% to about 40%, about 0% to about 50%, about 0% to about 75%, about 0% to about 90%, about 0% to about 95%, about 0% to about 99%, about 5% to about 10%, about 5% to about 15%, about 5% to about 20%, about 5% to about 30%, about 5% to about 40%, about 5% to about 50%, about 5% to about 75%, about 5% to about 90%, about 5% to about 95%, about 5% to about 99%, about 10% to about 15%, about 10% to about 20%, about 10% to about 30%, about 10% to about 40%, about 10% to about 50%, about 10% to about 75%, about 10% to about 90%, about 10% to about 95%, about 10% to about 99%, about 15% to about 20%, about 15% to about 30%, about 15% to about 40%, about 15% to about 50%, about 15% to about 75%, about 15% to about 90%, about 15% to about 95%, about 15% to about 99%, about 20% to about 30%, about 20% to about 40%, about 20% to about 50%, about 20% to about 75%, about 20% to about 90%, about 20% to about 95%, about 20% to about 99%, about 30% to about 40%, about 30% to about 50%, about 30% to about 75%, about 30% to about 90%, about 30% to about 95%, about 30% to about 99%, about 40% to about 50%, about 40% to about 75%, about 40% to about 90°/%, about 40% to about 95%, about 40% to about 99%, about 50% to about 75%, about 50% to about 90%, about 50% to about 95%, about 50% to about 99%, about 75% to about 90%, about 75% to about 95%, about 75% to about 99%, about 90% to about 95%, about 90% to about 99%, or about 95% to about 99%. In some embodiments, a formulation of a PLLBC of the disclosure has a loading capacity of about 0%, about 5%, about 10%, about 15%, about 20%, about 30%, about 40%, about 50%, about 75%, about 90%, about 95%, or about 99%. In some embodiments, a formulation of a PLLBC of the disclosure has a loading capacity of at least about 0%, about 5%, about 10%, about 15%, about 20%, about 30%, about 40%, about 50%, about 75%, about 90%, or about 95%. In some embodiments, a formulation of a PLLBC of the disclosure has a loading capacity of at most about 5%, about 10%, about 15%, about 20, about 30%, about 40%, about 50%, about 75%, about 90%, about 95%, or about 99%.

Prodrug Incorporation: Prodrug incorporation into PLLBCs of the disclosure such as liposomes, nanodroplets, or microbubbles can be measured in terms of moles of prodrug per moles of total phospholipid (mol %). In some embodiments, the prodrug incorporation into PLLBCs of the disclosure can be about 1 mol % to about 100 mol %. In some embodiments, the prodrug incorporation into PLLBCs of the disclosure can be about 1 mol % to about 10 mol %, about 1 mol % to about 20 mol %, about 1 mol % to about 30 mol %, about 1 mol % to about 40 mol %, about 1 mol % to about 50 mol %, about 1 mol % to about 60 mol %, about 1 mol % to about 70 mol %, about 1 mol % to about 80 mol %, about 1 mol % to about 90 mol %, about 1 mol % to about 100 mol %, about 10 mol % to about 20 mol %, about 10 mol % to about 30 mol %, about 10 mol % to about 40 mol %, about 10 mol % to about 50 mol %, about 10 mol % to about 60 mol %, about 10 mol % to about 70 mol %, about 10 mol % to about 80 mol %, about 10 mol % to about 90 mol %, about 10 mol % to about 100 mol %, about 20 mol % to about 30 mol %, about 20 mol % to about 40 mol %, about 20 mol % to about 50 mol %, about 20 mol % to about 60 mol %, about 20 mol % to about 70 mol %, about 20 mol % to about 80 mol %, about 20 mol % to about 90 mol %, about 20 mol % to about 100 mol %, about 30 mol % to about 40 mol %, about 30 mol % to about 50 mol %, about 30 mol % to about 60 mol %, about 30 mol % to about 70 mol %, about 30 mol % to about 80 mol %, about 30 mol % to about 90 mol %, about 30 mol % to about 100 mol %, about 40 mol % to about 50 mol %, about 40 mol % to about 60 mol %, about 40 mol % to about 70 mol %, about 40 mol % to about 80 mol %, about 40 mol % to about 90 mol %, about 40 mol % to about 100 mol %, about 50 mol % to about 60 mol %, about 50 mol % to about 70 mol %, about 50 mol % to about 80 mol %, about 50 mol % to about 90 mol %, about 50 mol % to about 100 mol %, about 60 mol % to about 70 mol %, about 60 mol % to about 80 mol %, about 60 mol % to about 90 mol %, about 60 mol % to about 100 mol %, about 70 mol % to about 80 mol %, about 70 mol % to about 90 mol %, about 70 mol % to about 100 mol %, about 80 mol % to about 90 mol %, about 80 mol % to about 100 mol %, or about 90 mol % to about 100 mol %. In some embodiments, the prodrug incorporation into PLLBCs of the disclosure can be about 1 mol %, about 10 mol %, about 20 mol %, about 30 mol %, about 40 mol %, about 50 mol %, about 60 mol %, about 70 mol %, about 80 mol %, about 90 mol %, or about 100 mol %. In some embodiments, the prodrug incorporation into PLLBCs of the disclosure can be at least about 1 mol %, about 10 mol %, about 20 mol %, about 30 mol %, about 40 mol %, about 50 mol %, about 60 mol %, about 70 mol %, about 80 mol %, or about 90 mol %. In some embodiments, the prodrug incorporation into PLLBCs of the disclosure can be at most about 10 mol %, about 20 mol %, about 30 mol %, about 40 mol %, about 50 mol %, about 60 mol %, about 70 mol %, about 80 mol %, about 90 mol %, or about 100 mol %.

Stability: The ability of PLLBCs such as liposomes, nanodroplets, or microbubbles to maintain a constant size is a measure of stability. In some embodiments PLLBCs of the disclosure (e.g. liposomes, nanodroplets, or microbubbles) are stable for about 1 day, about 2 days, about 3 days, about 4 days, about 5 days, about 6 days, about 7 days, about 8 days, about 9, days, about 10 days, about 11 days, about 12 days, about 13 days, about 14 days, 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 1 month, about 2 months, about 3 months, about 4 months, about 5 months, about 6 months, about 7 months, about 8 months, about 9 months, about 10 months, about 11 months, about 1 year, or about 5 years.

### Synthesis and Self-Assembly

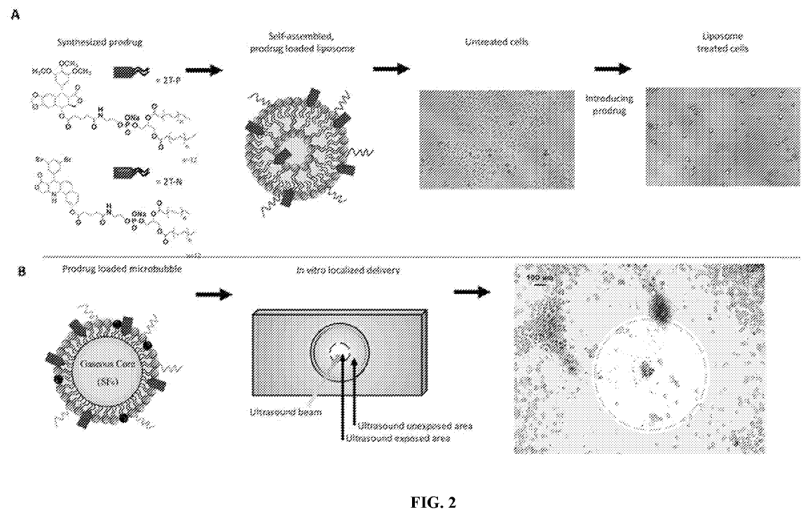

FIG. 2 presents non-limiting examples of PLLBCs. FIG. 2 PANEL A illustrates the synthesis of prodrugs, self-assembly of prodrug-loaded liposomes, and use of the liposomes for treating cells in vitro. FIG. 2 PANEL B shows the use of prodrug-loaded microbubbles and ultrasound exposure for targeted drug delivery in vitro.

### Mode of Administration

PLLBCs of the disclosure, such as liposomes and microbubbles can be administered in single or multiple doses to treat a condition. Administration of PLLBCs of the disclosures can occur by various forms and routes including, for example, intravenous, intra-arterial, subcutaneous, intramuscular, oral, parenteral, ophthalmic, subcutaneous, transdermal, nasal, vaginal, and topical administration. In some embodiments, PLLBCs of the disclosure can be administered locally, for example via injection directly into an organ.

### Amount and Frequency of Administration

Formulations of PLLBCs of the disclosure, such as liposomes and microbubbles, can be prepared in unit dosage forms suitable for single administration of precise dosages. In unit dosage form, formulations of PLLBCs of the disclosure are divided into unit doses containing appropriate quantities of prodrugs. The unit dosage can be in the form of a package containing discrete quantities of the formulation. Non-limiting examples are liquids in vials or ampoules.

Aqueous suspension compositions can be packaged in single-dose non-reclosable containers.

Multiple-dose reclosable containers can be used, for example, in combination with a preservative. Formulations for parenteral injection can be presented in unit dosage form, for example, in ampoules, or in multi-dose containers with a preservative.

PLLBCs of the disclosure can be present in unit dosage form in a formulation in a range of from about 0 mg/mL to about 400 mg/mL. PLLBCs of the disclosure can be present in unit dosage form in a formulation in a range of from about 0 mg/mL to about 5 mg/mL, about 0 mg/mL to about 10 mg/mL, about 0 mg/mL to about 15 mg/mL, about 0 mg/mL to about 20 mg/mL, about 0 mg/mL to about 25 mg/mL, about 0 mg/mL to about 50 mg/mL, about 0 mg/mL to about 75 mg/mL, about 0 mg/mL to about 100 mg/mL, about 0 mg/mL to about 200 mg/mL, about 0 mg/mL to about 300 mg/mL, about 0 mg/mL to about 400 mg/mL, about 5 mg/mL to about 10 mg/mL, about 5 mg/mL to about 15 mg/mL, about 5 mg/mL to about 20 mg/mL, about 5 mg/mL to about 25 mg/mL, about 5 mg/mL to about 50 mg/mL, about 5 mg/mL to about 75 mg/mL, about 5 mg/mL to about 100 mg/mL, about 5 mg/mL to about 200 mg/mL, about 5 mg/mL to about 300 mg/mL, about 5 mg/mL to about 400 mg/mL, about 10 mg/mL to about 15 mg/mL, about 10 mg/mL to about 20 mg/mL, about 10 mg/mL to about 25 mg/mL, about 10 mg/mL to about 50 mg/mL, about 10 mg/mL to about 75 mg/mL, about 10 mg/mL to about 100 mg/mL, about 10 mg/mL to about 200 mg/mL, about 10 mg/mL to about 300 mg/mL, about 10 mg/mL to about 400 mg/mL, about 15 mg/mL to about 20 mg/mL, about 15 mg/mL to about 25 mg/mL, about 15 mg/mL to about 50 mg/mL, about 15 mg/mL to about 75 mg/mL, about 15 mg/mL to about 100 mg/mL, about 15 mg/mL to about 200 mg/mL, about 15 mg/mL to about 300 mg/mL, about 15 mg/mL to about 400 mg/mL, about 20 mg/mL to about 25 mg/mL, about 20 mg/mL to about 50 mg/mL, about 20 mg/mL to about 75 mg/mL, about 20 mg/mL to about 100 mg/mL, about 20 mg/mL to about 200 mg/mL, about 20 mg/mL to about 300 mg/mL, about 20 mg/mL to about 400 mg/mL, about 25 mg/mL to about 50 mg/mL, about 25 mg/mL to about 75 mg/mL, about 25 mg/mL to about 100 mg/mL, about 25 mg/mL to about 200 mg/mL, about 25 mg/mL to about 300 mg/mL, about 25 mg/mL to about 400 mg/mL, about 50 mg/mL to about 75 mg/mL, about 50 mg/mL to about 100 mg/mL, about 50 mg/mL to about 200 mg/mL, about 50 mg/mL to about 300 mg/mL, about 50 mg/mL to about 400 mg/mL, about 75 mg/mL to about 100 mg/mL, about 75 mg/mL to about 200 mg/mL, about 75 mg/mL to about 300 mg/mL, about 75 mg/mL to about 400 mg/mL, about 100 mg/mL to about 200 mg/mL, about 100 mg/mL to about 300 mg/mL, about 100 mg/mL to about 400 mg/mL, about 200 mg/mL to about 300 mg/mL, about 200 mg/mL to about 400 mg/mL, or about 300 mg/mL to about 400 mg/mL. PLLBCs of the disclosure can be present in unit dosage form in a formulation in a range of from about 0 mg/mL, about 5 mg/mL, about 10 mg/mL, about 15 mg/mL, about 20 mg/mL, about 25 mg/mL, about 50 mg/mL, about 75 mg/mL, about 100 mg/mL, about 200 mg/mL, about 300 mg/mL, or about 400 mg/mL. PLLBCs of the disclosure can be present in unit dosage form in a formulation in a range of from at least about 0 mg/mL, about 5 mg/mL, about 10 mg/mL, about 15 mg/mL, about 20 mg/mL, about 25 mg/mL, about 50 mg/mL, about 75 mg/mL, about 100 mg/mL, about 200 mg/mL, or about 300 mg/mL. PLLBCs of the disclosure can be present in a unit dosage form in a formulation in a range of from at most about 5 mg/mL, about 10 mg/mL, about 15 mg/mL, about 20 mg/mL, about 25 mg/mL, about 50 mg/mL, about 75 mg/mL, about 100 mg/mL, about 200 mg/mL, about 300 mg/mL, or about 400 mg/mL.

The dosage level of PLLBCs of the disclosure such as liposomes, nanodroplets, or microbubbles can depend upon a variety of factors including, for example, the activity of the PLLBC employed, the route of administration, the time of administration, the rate of excretion, metabolism of the prodrug, the duration of the treatment, prodrug compound, compounds and/or materials used in combination with the PLLBCs, the age, sex, weight, condition, general health, and prior medical history of the subject being treated. The dosage values can also vary with the severity of the condition to be treated. For any particular subject, specific dosage regimens can be adjusted over time according to the individual need and the professional judgment of the person administering or supervising the administration of the compositions.

In some embodiments, a dose can be expressed in terms of an amount of prodrug divided by the mass of a subject, for example, milligrams of prodrug per kilograms of subject body mass. In some embodiments, a dose is administered in an amount ranging from about 5 mg/kg to about 50 mg/kg, 250 mg/kg to about 2000 mg/kg, about 10 mg/kg to about 800 mg/kg, about 50 mg/kg to about 400 mg/kg, about 100 mg/kg to about 300 mg/kg, or about 150 mg/kg to about 200 mg/kg.

In some embodiments, a dose can be expressed in terms of an amount of PLLBC (e.g. liposomes, nanodroplets, or microbubbles) per kilograms of subject body mass. In some embodiments, a dose is administered in an amount ranging from about 5 mg/kg to about 50 mg/kg, 250 mg/kg to about 2000 mg/kg, about 10 mg/kg to about 800 mg/kg, about 50 mg/kg to about 400 mg/kg, about 100 mg/kg to about 300 mg/kg, or about 150 mg/kg to about 200 mg/kg.

### Combination Treatments

In some embodiments, PLLBCs of the disclosure comprise multiple prodrugs. Administration of PLLBCs comprising combinations of prodrugs can have a synergistic effect. Synergy can refer to the observation that administration of PLLBCs comprising multiple prodrugs can have an overall effect that is greater than the sum of the individual effect of administering PLLBCs containing a single prodrug. Synergy can also refer to the observation that a PLLBC with a single prodrug produces little or no effect but, a PLLBC with multiple prodrugs produces an effect that is greater than the effect produced by the PLLBC with the second prodrug alone. Synergy can also refer to the observation that the administration of PLLBCs with multiple prodrugs to a subject reduces side effects in the subject compared to the administration of a PLLBC with a single prodrug.

In some embodiments, lipid based carriers loaded with different prodrugs can be administered in combination to a subject. Administration of a combination of PLLBCs (e.g. liposomes, nanodroplets, or microbubbles) can have a synergistic effect. Synergy can refer to the observation that the combination of two prodrug loaded lipid-based carriers can have an overall effect that is greater than the sum of the two individual effects. Synergy can also refer to the observation that a single PLLBC produces little or no effect but, when administered with a second PLLBC produces an effect that is greater than the effect produced by the second PLLBC alone. Synergy can also refer to the observation that the administration of two PLLBCs to a subject in combination reduces side effects in the subject compared to the administration of a PLLBC alone. Administration of different PLLBCs can occur simultaneously or sequentially through the same or different routes of administration.

### Triggering of Prodrug Activity at Target Sites

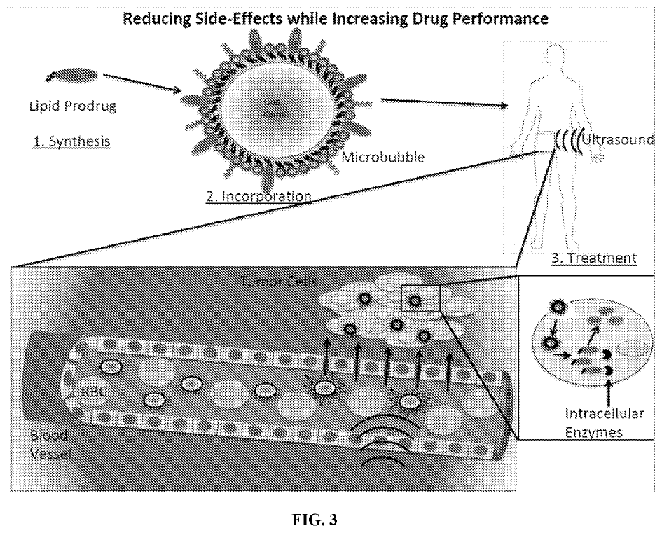

In some embodiments, PLLBCs of the disclosure such as liposomes, nanodroplets, or microbubbles can deliver prodrugs to a target site in a subject. A target site can be, for example, a site of localized infection, a cancerous lesion, a non-cancerous lesion, a metastatic lesion, a pre-cancerous lesion, a tumor, an organ, or a specific a cell-type such as red blood cells, white blood cells, neutrophils, macrophages, or neurons. Delivery of prodrugs to a target site can, for example, decrease the dose needed to effectively treat a condition, increase the effectiveness of a prodrug, or decrease side effects caused by the prodrug in a subject. In some embodiments, prodrugs present at the target site as part of PLLBCs can remain inactive in the absence of an extracorporeal trigger. Upon the presence of an extracorporeal trigger, the PLLBC can be triggered causing the prodrug to be released and activated. Non limiting examples of extracorporeal triggers include ultrasound, magnetic fields, electric fields, light waves, and radiation. A schematic representation of treatment with a microbubble utilizing ultrasound as an extracorporeal trigger is shown in FIG. 3

Ultrasound is sound waves with frequencies higher than the upper audible limit of human hearing. In some embodiments, a PLLBC of the disclosure (e.g. liposomes, microbubbles, or nanodroplets) can be triggered using ultrasound frequencies of about 1 MHz to about 20 MHz. In some embodiments, a PLLBCs of the disclosure can be triggered using ultrasound frequencies of about 1 MHz to about 2 MHz, about 1 MHz to about 3 MHz, about 1 MHz to about 4 MHz, about 1 MHz to about 5 MHz, about 1 MHz to about 6 MHz, about 1 MHz to about 7 MHz, about 1 MHz to about 8 MHz, about 1 MHz to about 9 MHz, about 1 MHz to about 10 MHz, about 1 MHz to about 15 MHz, about 1 MHz to about 20 MHz, about 2 MHz to about 3 MHz, about 2 MHz to about 4 MHz, about 2 MHz to about 5 MHz, about 2 MHz to about 6 MHz, about 2 MHz to about 7 MHz, about 2 MHz to about 8 MHz, about 2 MHz to about 9 MHz, about 2 MHz to about 10 MHz, about 2 MHz to about 15 MHz, about 2 MHz to about 20 MHz, about 3 MHz to about 4 MHz, about 3 MHz to about 5 MHz, about 3 MHz to about 6 MHz, about 3 MHz to about 7 MHz, about 3 MHz to about 8 MHz, about 3 MHz to about 9 MHz, about 3 MHz to about 10 MHz, about 3 MHz to about 15 MHz, about 3 MHz to about 20 MHz, about 4 MHz to about 5 MHz, about 4 MHz to about 6 MHz, about 4 MHz to about 7 MHz, about 4 MHz to about 8 MHz, about 4 MHz to about 9 MHz, about 4 MHz to about 10 MHz, about 4 MHz to about 15 MHz, about 4 MHz to about 20 MHz, about 5 MHz to about 6 MHz, about 5 MHz to about 7 MHz, about 5 MHz to about 8 MHz, about 5 MHz to about 9 MHz, about 5 MHz to about 10 MHz, about 5 MHz to about 15 MHz, about 5 MHz to about 20 MHz, about 6 MHz to about 7 MHz, about 6 MHz to about 8 MHz, about 6 MHz to about 9 MHz, about 6 MHz to about 10 MHz, about 6 MHz to about 15 MHz, about 6 MHz to about 20 MHz, about 7 MHz to about 8 MHz, about 7 MHz to about 9 MHz, about 7 MHz to about 10 MHz, about 7 MHz to about 15 MHz, about 7 MHz to about 20 MHz, about 8 MHz to about 9 MHz, about 8 MHz to about 10 MHz, about 8 MHz to about 15 MHz, about 8 MHz to about 20 MHz, about 9 MHz to about 10 MHz, about 9 MHz to about 15 MHz, about 9 MHz to about 20 MHz, about 10 MHz to about 15 MHz, about 10 MHz to about 20 MHz, or about 15 MHz to about 20 MHz. In some embodiments, a PLLBCs of the disclosure can be triggered using ultrasound frequencies of about 1 MHz, about 2 MHz, about 3 MHz, about 4 MHz, about 5 MHz, about 6 MHz, about 7 MHz, about 8 MHz, about 9 MHz, about 10 MHz, about 15 MHz, or about 20 MHz. In some embodiments, a PLLBCs of the disclosure can be triggered using ultrasound frequencies of at least about 1 MHz, about 2 MHz, about 3 MHz, about 4 MHz, about 5 MHz, about 6 MHz, about 7 MHz, about 8 MHz, about 9 MHz, about 10 MHz, or about 15 MHz. In some embodiments, a PLLBC of the disclosure can be triggered using ultrasound frequencies of at most about 2 MHz, about 3 MHz, about 4 MHz, about 5 MHz, about 6 MHz, about 7 MHz, about 8 MHz, about 9 MHz, about 10 MHz, about 15 MHz, or about 20 MHz.

In some embodiments, ultrasound waves can produce pressure that acts on PLLBCs. In some embodiments, this pressure is about 25 kPa to about 2.5 MPa of pressure. In some embodiments, the pressure is about 25 kPa to about 300 kPa of pressure. In some embodiments, the pressure is about 1 Mpa to about 2.5 MPa of pressure.

In some embodiments, light can be used to trigger a PLLBC such as a liposome, nanodroplet, or microbubble. In some embodiments, the light is in the form of a laser pulse. In some embodiments, the wavelength of light can be about 400 nm to about 1,400 nm. In some embodiments, the wavelength of light can be about 400 nm to about 450 nm, about 400 nm to about 500 nm, about 400 nm to about 550 nm, about 400 nm to about 600 nm, about 400 nm to about 650 nm, about 400 nm to about 700 nm, about 400 nm to about 750 nm, about 400 nm to about 800 nm, about 400 nm to about 900 nm, about 400 nm to about 1,000 nm, about 400 nm to about 1,400 nm, about 450 nm to about 500 nm, about 450 nm to about 550 nm, about 450 nm to about 600 nm, about 450 nm to about 650 nm, about 450 nm to about 700 nm, about 450 nm to about 750 nm, about 450 nm to about 800 nm, about 450 nm to about 900 nm, about 450 nm to about 1,000 nm, about 450 nm to about 1,400 nm, about 500 nm to about 550 nm, about 500 nm to about 600 nm, about 500 nm to about 650 nm, about 500 nm to about 700 nm, about 500 nm to about 750 nm, about 500 nm to about 800 nm, about 500 nm to about 900 nm, about 500 nm to about 1,000 nm, about 500 nm to about 1,400 nm, about 550 nm to about 600 nm, about 550 nm to about 650 nm, about 550 nm to about 700 nm, about 550 nm to about 750 nm, about 550 nm to about 800 nm, about 550 nm to about 900 nm, about 550 nm to about 1,000 nm, about 550 nm to about 1,400 nm, about 600 nm to about 650 nm, about 600 nm to about 700 nm, about 600 nm to about 750 nm, about 600 nm to about 800 nm, about 600 nm to about 900 nm, about 600 nm to about 1,000 nm, about 600 nm to about 1,400 nm, about 650 nm to about 700 nm, about 650 nm to about 750 nm, about 650 nm to about 800 nm, about 650 nm to about 900 nm, about 650 nm to about 1,000 nm, about 650 nm to about 1,400 nm, about 700 nm to about 750 nm, about 700 nm to about 800 nm, about 700 nm to about 900 nm, about 700 nm to about 1,000 nm, about 700 nm to about 1,400 nm, about 750 nm to about 800 nm, about 750 nm to about 900 nm, about 750 nm to about 1,000 nm, about 750 nm to about 1,400 nm, about 800 nm to about 900 nm, about 800 nm to about 1,000 nm, about 800 nm to about 1,400 nm, about 900 nm to about 1,000 nm, about 900 nm to about 1,400 nm, or about 1,000 nm to about 1,400 nm. In some embodiments, the wavelength of light can be about 400 nm, about 450 nm, about 500 nm, about 550 nm, about 600 nm, about 650 nm, about 700 nm, about 750 nm, about 800 nm, about 900 nm, about 1,000 nm, or about 1,400 nm. In some embodiments, the wavelength of light can be at least about 400 nm, about 450 nm, about 500 nm, about 550 nm, about 600 nm, about 650 nm, about 700 nm, about 750 nm, about 800 nm, about 900 nm, or about 1,000 nm. In some embodiments, the wavelength of light can be at most about 450 nm, about 500 nm, about 550 nm, about 600 nm, about 650 nm, about 700 nm, about 750 nm, about 800 nm, about 900 nm, about 1,000 nm, or about 1,400 nm.

In some embodiments, a magnetic field can be used to trigger a PLLBC such as a liposome, nanodroplet, or microbubble. In some embodiments, the magnetic field strength is 0.2 T to about 7 T. In some embodiments, the magnetic field strength is about 0.2 T to about 0.5 T, about 0.2 T to about 1 T, about 0.2 T to about 1.5 T, about 0.2 T to about 2 T, about 0.2 T to about 3 T, about 0.2 T to about 4 T, about 0.2 T to about 5 T, about 0.2 T to about 6 T, about 0.2 T to about 7 T, about 0.5 T to about 1 T, about 0.5 T to about 1.5 T, about 0.5 T to about 2 T, about 0.5 T to about 3 T, about 0.5 T to about 4 T, about 0.5 T to about 5 T, about 0.5 T to about 6 T, about 0.5 T to about 7 T, about 1 T to about 1.5 T, about 1 T to about 2 T, about 1 T to about 3 T, about 1 T to about 4 T, about 1 T to about 5 T, about 1 T to about 6 T, about 1 T to about 7 T, about 1.5 T to about 2 T, about 1.5 T to about 3 T, about 1.5 T to about 4 T, about 1.5 T to about 5 T, about 1.5 T to about 6 T, about 1.5 T to about 7 T, about 2 T to about 3 T, about 2 T to about 4 T, about 2 T to about 5 T, about 2 T to about 6 T, about 2 T to about 7 T, about 3 T to about 4 T, about 3 T to about 5 T, about 3 T to about 6 T, about 3 T to about 7 T, about 4 T to about 5 T, about 4 T to about 6 T, about 4 T to about 7 T, about 5 T to about 6 T, about 5 T to about 7 T, or about 6 T to about 7 T. In some embodiments, the magnetic field strength is about 0.2 T, about 0.5 T, about 1 T, about 1.5 T, about 2 T, about 3 T, about 4 T, about 5 T, about 6 T, or about 7 T. In some embodiments, the magnetic field strength is at least about 0.2 T, about 0.5 T, about 1 T, about 1.5 T, about 2 T, about 3 T, about 4 T, about 5 T. or about 6 T. In some embodiments, the magnetic field strength is at most about 0.5 T, about 1 T, about 1.5 T, about 2 T, about 3 T, about 4 T, about 5 T, about 6 T, or about 7 T.

The amount of time in which an extracorporeal trigger is applied to a subject can vary. In some embodiments the extracorporeal trigger is applied for about 1 second, about 2 seconds about 3 seconds, about 4 seconds, about 5 seconds, about 10 seconds, about 30 seconds, about 1 minute, about 5 minutes, about 15 minutes, about 30 minutes, about 1 hour, about 2 hours, about 3 hours, about 4 hours, about 5 hours, about 6 hours or about 12 hours. In some embodiments, an extracorporeal trigger is applied for about 1 second to about 30 seconds, about 1 minute to about 30 minutes, or about 1 hour to about 6 hours, or about 6 hours to about 12 hours.

In some embodiments, an extracorporeal trigger is applied to a subject in pulses. In some embodiments an extracorporeal trigger is applied in 2-100 pulses. In some embodiments an extracorporeal trigger can be applied in 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 pulses. In some embodiments, an extracorporeal trigger is applied in more than 100 pulses. In some embodiments each pulse of an extracorporeal trigger is applied for about 1 second, about 2 seconds about 3 seconds, about 4 seconds, about 5 seconds, about 10 seconds, about 30 seconds, about 1 minute, about 5 minutes, about 15 minutes, about 30 minutes, about 1 hour, about 2 hours, about 3 hours, about 4 hours, about 5 hours, about 6 hours or about 12 hours. In some embodiments a pulse of an extracorporeal trigger is applied for about 1 second to about 30 seconds, about 1 minute to about 30 minutes, or about 1 hour to about 6 hours, or about 6 hours to about 12 hours.

### Contrast Agents

In some embodiments, PLLBCs of the disclosure such as liposomes, nanodroplets, or microbubbles can act as contrast agents. Contrast agents can improve imaging techniques such as ultrasound imaging. Ultrasound imaging is portable, provides real-time imaging feedback, and lacks ionizing radiation risks. Ultrasound contrast agents, such as microbubbles, respond non-linearly to ultrasound and provide a high signal-to-noise ratio for imaging in cardiology. The ability of ultrasound to visualize targeted tissue areas with microbubbles in real-time can allow the measurement of, for example, tumor dimensions, vasculature, and blood flow.

### Treatment of Conditions

PLLBCs, such as liposomes, nanodroplets, or microbubbles, of the disclosure can be used to treat, prevent, or diagnose a condition. In some embodiments, PLLBCs of the disclosure can be used to treat, prevent, or diagnose a condition, for example, cancer, a viral infection, a bacterial infection, inflammatory disorders, or neurological disorders.

PLLBCs (e.g. liposomes, nanodroplets, or microbubbles) of the disclosure can be used to treat, prevent, or diagnose cancer. In some embodiments, the PLLBCs of the disclosure can be used to treat cancer, for example, acute lymphoblastic leukemia (ALL), acute myeloid leukemia (AML), adrenocortical carcinoma, Kaposi sarcoma (soft tissue sarcoma), AIDS-related lymphoma, primary central nervous system lymphoma, anal cancer, gastrointestinal carcinoid tumors, astrocytoma, atypical teratoid/rhabdoid tumors, basal cell carcinoma, bile duct cancer, bladder cancer, bone cancer, brain tumors, breast cancer, bronchial tumors, Burkitt lymphoma, non-Hodgkin lymphoma, carcinoid tumors, cardiac tumors, embryonal tumors, germ cell tumors, cervical cancer, cholangiocarcinoma, chordoma, chronic lymphocytic leukemia (CLL), chronic myelogenous leukemia (CML), chronic myeloproliferative neoplasms, colorectal cancer, craniopharyngioma, cutaneous T-cell lymphoma, ductal carcinoma in situ (DCIS), endometrial cancer (uterine cancer), ependymoma, esophageal cancer, esthesioneuroblastoma (head and neck cancer), Ewing sarcoma, extracranial germ cell tumors, intraocular melanoma, retinoblastoma, fallopian tube cancer, fibrous histiocytoma of bone, osteosarcoma, gall bladder cancer, gastrointestinal carcinoid tumors, gastrointestinal stromal tumors (GIST), extra gonadal germ cell tumors, ovarian germ cell tumors, testicular cancer, gestational trophoblastic disease, Hairy cell leukemia, head and neck cancer, hepatocellular (liver) cancer, histiocytosis, hypopharyngeal cancer, intraocular melanoma, islet cell tumors, pancreatic neuroendocrine tumors, kidney (renal cell) cancer, Langerhans cell histiocytosis, laryngeal cancer, lip and oral cavity cancer, liver cancer, non-small cell lung cancer, small cell lung cancer, lymphoma, malignant fibrous histiocytoma of bone, osteosarcoma, melanoma, Merkel cell carcinoma, mesothelioma, metastatic cancer, metastatic squamous neck cancer, midline tract carcinoma, mouth cancer, multiple endocrine neoplasia syndromes, multiple myeloma, mycosis fungoid (lymphoma), nasal cavity and paranasal sinus cancer, neuroblastoma, non-Hodgkin lymphoma, pancreatic cancer, pancreatic neuroendocrine tumors, papillomatosis, paraganglioma, parathyroid cancer, penile cancer, pharyngeal cancer, pheochromocytoma, pituitary tumors, pleuropulmonary blastoma, primary peritoneal cancer, prostate cancer, rectal cancer, rhabdomyosarcoma, salivary gland cancer, vascular tumors, Sezary syndrome (lymphoma), small intestine cancer, soft tissue sarcoma, T-cell lymphoma, testicular cancer, throat cancer, nasopharyngeal cancer, oropharyngeal cancer, hypopharyngeal cancer, thymoma and thymic carcinoma, thyroid cancer, urethral cancer, uterine sarcoma, vaginal cancer, vascular tumors, vulvar cancer, or Wilms tumors.

PLLBCs (e.g. liposomes, nanodroplets, or microbubbles) of the disclosure can be used to treat, prevent, or diagnose a viral infection. In some embodiments, the PLLBCs of the disclosure can be used to treat a respiratory viral infection, such as the flu, a respiratory syncytial viral infection, adenovirus infection, parainfluenza viral infection, or severe acute respiratory syndrome (SARS). In some embodiments, PLLBCs of the disclosure can be used to treat a gastrointestinal viral infection, for example, a norovirus infection, rotavirus infection, adenovirus infection, or astrovirus infections. In some embodiments, PLLBCs of the disclosure of the disclosure can be used to treat an exanthematous viral infection, for example, measles, rubella, chickenpox, shingles, roseola, smallpox, fifth disease, or a chikungunya viral infection.

In some embodiments, the liposomes, nanodroplets, or microbubbles of the disclosure can be used to treat a hepatic viral infection, such as hepatitis A, hepatitis B, hepatitis C, hepatitis D, or hepatitis E. In some embodiments, PLLBCs of the disclosure of the disclosure can be used to treat a cutaneous viral infection, for example, warts (e.g., genital warts), oral herpes, genital herpes, or molluscum contagiosum. In some embodiments, PLLBCs of the disclosure of the disclosure can be used to treat a hemorrahagic viral disease, for example, Ebola, Lassa fever, dengue fever, yellow fever, Marburg hemorrhagic fever, or Crimean-Congo hemorrhagic fever.

In some embodiments, PLLBCs of the disclosure of the disclosure can be used to treat a neurologic viral infection, for example, polio, viral meningitis, viral encephalitis, or rabies.

PLLBCs (e.g. liposomes, nanodroplets, or microbubbles) of the disclosure can be used to treat, prevent, or diagnose a bacterial infection. In some embodiments, PLLBCs of the disclosure can be used to treat or prevent bacterial infections, for example, Staphylococcus aureus, Staphylococcus epidermis, Staphylococcus saprophyticus, Streptococcus pyogenous, Streptococcus agalacliae, Streptococcus bovis, Streptococcus pneumoniae, Viridians streptococci, Bacillus anthracis, Bacillus cereus, Clostridium tetani, Clostridium botulimum, Clostridium perfringens, Clostridium dificile, Corynebacterium diphtheriae, Listeria monocytogenes, Neisseria menigitidis, Neisseria gonorrhoeae, Escherichia coli, Salmonella typhi, Shigella (bacillary dysentery), Vibrio cholerae, Campylobacter jejuni, or Helicobacter pylori.

### EXAMPLES

### Example 1: Synthetic Methods

SCHEME 1 describes the synthetic route used to couple two chemotherapeutic compounds (denoted P and N) to a linker containing two phospholipid chains.

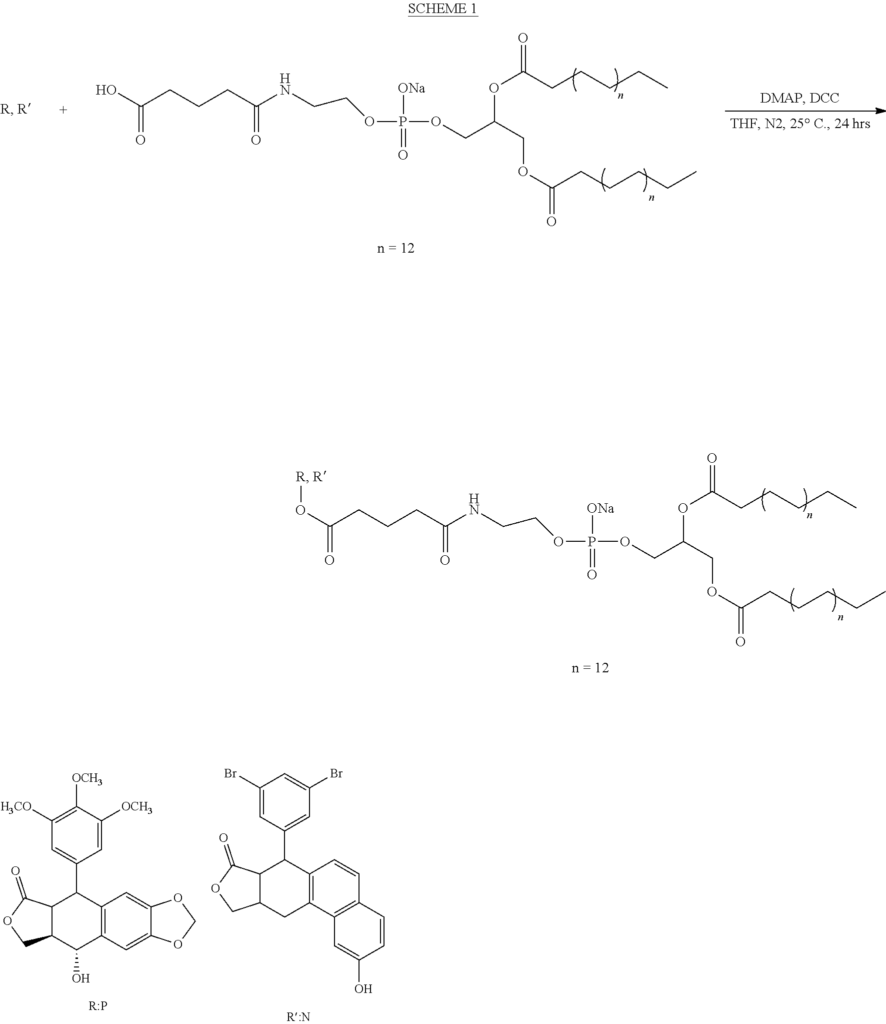

A mixture of parent compound (0.24 mmol, 1 eq.), DCC (0.73 mmol, 3 eq.), DPPE-Glu (0.24 mmol, 1 eq.) and DMAP (0.048 mmol, 0.4 eq.) was added in a 10 mL flask. 5.5 mL of dry THF was added to the flask under a nitrogen atmosphere. The reaction mixture was stirred at room temperature for 24 hours. Thin layer chromatography (TLC) plates were used to monitor the reactions and guide all flash column chromatography (Kiesel gel 60, 230-400 mesh). High resolution mass spectrometry was used to verify the chemical structures of the prodrugs. The hydrophobic fatty acid of each prodrug served as an anchor to incorporate within the lipid layers of lipophilic drug delivery vehicles.

Sodium 2,3-bis (palmitoyloxy) propyl (2-(5-oxo-5-(((5S,5aS,8aS,9S)-8-oxo-9-(3,4,5-trimethoxyphenyl)-5,5a,6,8,8a,9-hexahydrofuro[3′,4′:6,7]naphtha[2,3-d][1,3]dioxol-5-yl) oxy) pentanamido)ethyl)phosphate (2T-P). 28.6% yield as white solid, mp=60° C. (CH₂Cl₂/MeOH=7/1). ¹H NMR (CDCl₃-d₆) 6.83 (s, 1H), 6.53 (s, 1H), 6.39 (s, 2H), 5.98 (s, 2H), 5.91 (d, J=7.04 Hz, 1H), 5.26 (s, 1H), 4.58 (d, J=3.32, 1H), 4.37 (s, 2H), 4.23-4.15 (m, 2H), 3.94 (s, 4H), 3.79 (d, J=19.2 Hz, 9H), 3.50 (s, 2H), 2.89-2.82 (m, 2H), 2.56-2.50 (m, 2H), 2.35-2.19 (m, 6H), 2.12-2.01 (m, 7H), 1.31 (s, 48H), 0.91-0.88 (m, 6H); ¹³C NMR (CDCl₃-d₆) 173.8, 152.6, 148.1, 147.5, 134.9, 132.4, 108.2, 101.7, 60.7, 56.2, 38.7, 34.5, 34.3, 33.5, 31.9, 29.8, 29.7, 29.4, 25, 24.9, 22.7, 14.1.

Sodium 2,3-bis(palmitoyloxy)propyl(2-(5-((7-(3,5-dibromophenyl)-8-oxo-7,8,10,11-tetrahydrobenzo[h]furo[3,4-b]quinolin-2-yl)oxy)-5-oxopentanamido)ethyl) phosphate (2T-N). 25.6% yield as oil, (CH₂Cl₂/MeOH=7/1). ¹H NMR (CDCl₃-d₆) 10.56 (s, 1H), 8.06 (s, 1H), 7.89 (d, J=6.88 Hz, 1H); 7.72 (d, J=8.96 Hz, 1H), 7.59 (s, 1H), 7.44 (s, 1H), 7.37-7.29 (m, 3H), 7.19 (d, J=8.8 Hz, 1H), 6.96 (d, J=8.36, 1H), 6.38 (d, J=7.08 Hz, 1H), 5.25 (s, 1H), 5.13-4.99 (m, 3H), 4.34 (d, J=, 1H), 4.02-4.01 (m, 5H), 3.51 (d, J=14.08 Hz, 2H), 3.05 (s, 3H), 2.69 (s, 2H), 2.41 (s, 2H), 2.23 (d, J=6 Hz, 4H), 2.09 (s, 2H), 1.31-1.21 (m, 46H) 0.91-0.89 (m, 6H); ¹³C NMR (CDCl₃-d₆) 173.6, 173.4, 173.2, 173.2, 172.4 158.9, 156.6, 149.8, 148.8, 139.7, 132.4, 131.9, 131.2, 130.2, 129.6, 128.0, 123.7, 123.1, 121.6, 118.6, 106.4, 96.1, 66.5, 64.5, 62.5, 40.9, 39.8, 34.9, 34.2, 34.0, 32.7, 31.9, 29.7, 29.7, 29.6, 29.6, 29.4, 29.4, 29.2, 29.1, 24.9, 24.8, 22.7, 20.9, 14.1.

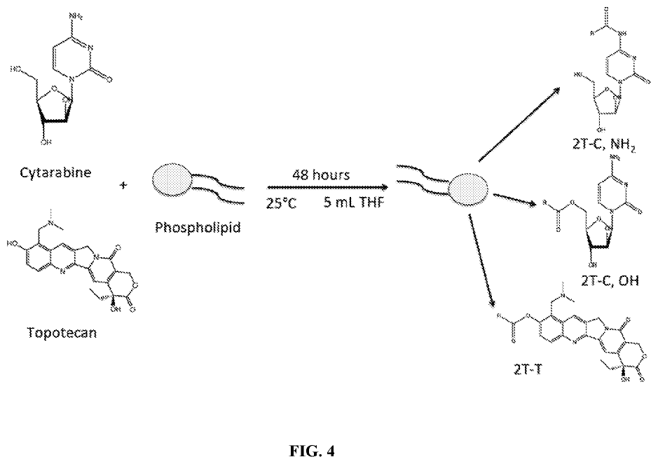

FIG. 1 and FIG. 4 illustrate the synthetic routes used to couple topotecan and cytarabine to phospholipids. Phospholipid conjugation was carried out either through an amino attachment (cytarabine) or a hydroxyl attachment (cytarabine, topotecan) to produce two tailed topotecan (2T-T) or two tailed cytarabine (2T-C). Reaction progress was checked using thin layer chromatography and prodrugs were purified through chromatographic separation using 3:1 CH₂Cl₂:MeOH (2T-C, NH₂), 1:1 CH₂Cl₂:MeOH (2T-C, OH), and 4:1 CHCl₃:MeOH (2T-T). Structures of the prodrugs were verified through ¹H and C¹³ nuclear magnetic resonance spectroscopy (NMR), and yield was found to be 23% for 2T-C (amino), 7% for 2T-C(OH), and 19% for 2T-T.

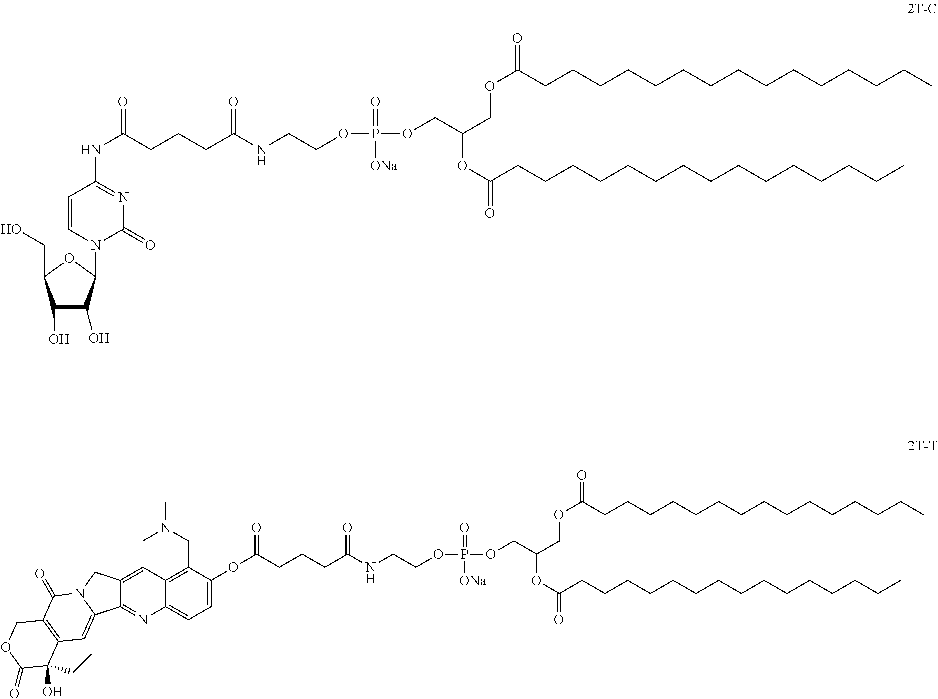

### Example 2: Liposome Suspension

Liposome prodrug-loaded lipid films were prepared using a chloroform solution of 1,2-dipalmitoyl-sn-glycero-3-phosphocholine (DPPC); 1,2-distearoyl-sn-glycero-3-phosphoethanolamine-N-(methoxy (polyethyleneglycol) 2000) ammonium salt (DSPE-PEG2000); and a prodrug solution in chloroform at a desired mol %. The lipid mixture was then dried under nitrogen gas and further under vacuum at 50° C. for 2 h. The prodrug-enriched lipid films were resuspended in 0.5 mL aliquots of a 1× phosphate buffer saline (PBS) solution using a sonication bath for 30 min at 50° C. to provide 1 mg of lipid per 1 mL of PBS liposome suspension.

### Example 3: Microbubble Suspension

To produce 2T-N loaded microbubbles, 2T-N prodrug-loaded lipid films were produced using a chloroform solution of 1,2-dipalmitoly-sn-glycero-3-phosphocholine (DPPC); 1,2-dipalmitoyl-sn-glycero-3-phophate (monosodium salt) (DPPA); 1,2-distearoyl-sn-glycero-3-phosphoethanolnamine-N-(folate (polyethylene glycol)-5000) (ammonium salt) (DSPE-PEG5000); and a prodrug solution in chloroform at a desired mol %. The lipid mixture was then dried under nitrogen gas and further dried under vacuum at 50° C. for 2 h. The prodrug-enriched lipid film was resuspended in 1.5 mL aliquots of (80 vol % 0.1 M Tris, 10 vol % glycerin, 10 vol % propylene glycol) Tris buffer using a sonication bath for 30 min at 50° C., resulting in a 1.5 mg of lipids per 1.5 mL of Tris buffer liposome suspension. Post-sealing, each vial was purged with 10 mL of sulfur hexafluoride (SF₆). A mechanical shaker was used to shake the vials for 45 seconds to form microbubbles from the liposome suspension.

### Example 4: Nanodroplet Suspension

DSPC:DSPE-PEG2000 were formed and used to generate nanodroplets (NDs) with perfluorobutane. The microbubble containing vial was submerged in a 5° C. CO₂/isopropanol bath and vented with a 27 G needle then pressurized with approximately 30 mL of air (from room). Freezing of the lipids was prevented by observing the vial contents and temperature of the bath.

### Example 5: Differential Scanning Calorimetry

Prodrug-loaded liposome (PLL) samples were prepared using 20 mg of lipid per 1 mL of PBS for each compound with increasing prodrug concentrations without extrusion. Deionized water was used as the calibration standard. 10 μL from each liposome suspension was transferred and sealed in an aluminum DSC pan. DSC measurements were taken at room temperature, and the sample was then heated from 15° C. to 55° C. at 5° C./min.

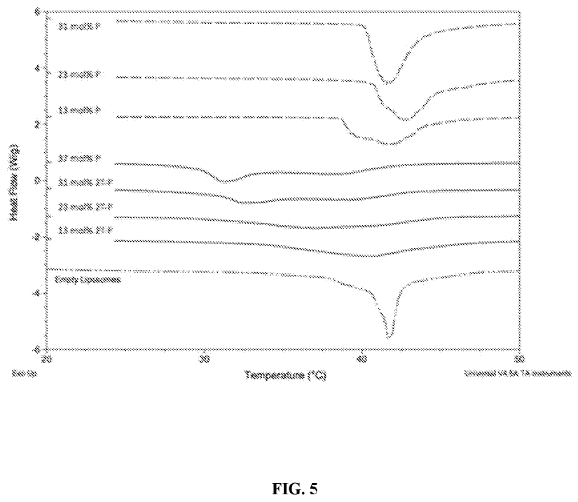

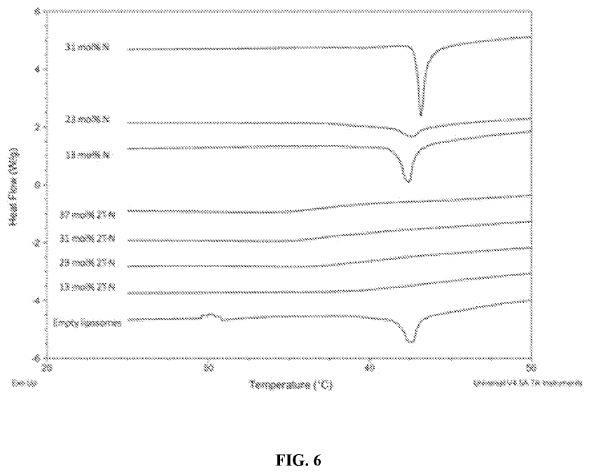

FIG. 5 shows differential scanning calorimetry curves of 2T-P-loaded liposomes with increasing concentrations. FIG. 6 shows differential scanning calorimetry curves of P-loaded liposomes with increasing concentrations.

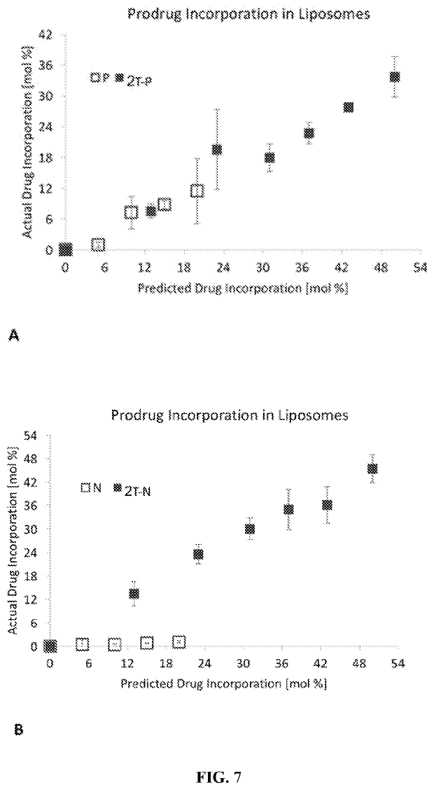

DSC is a thermal analytical technique useful for designing lipid drug delivery systems, like liposomes, by determining the compatibility of mixed molecules and compositions with the liposome bilayer. The thermotropic behavior of the liposomes at varying prodrug concentrations is measured to determine whether insertion of the molecules modifies the phase transition temperature. The pre-phase transition was masked by PEG-2000. Compared to empty liposomes, PLL (2T-P and 2T-N; 0 to 37 mol %) thermograms showed shallow decreasing phase transition temperatures with increasing prodrug compositions. P-loaded and N-loaded liposome (0 to 31 mol %) thermograms maintained the phase transition temperature with little to no decrease in endothermic peaks. The decreases in the endothermic peaks of 2T-P- and 2T-N-loaded liposomes demonstrated changes in the lipid-lipid interactions resulting from the presence of the prodrugs in the bilayer. P resulted in a slight change in the phase transition at 13 mol % corresponding to the drug incorporation (FIG. 7 PANEL A and FIG. 7 PANEL B). As the concentration was increased, the phase transition returned to the phase transition of empty liposomes. A higher concentration of P resulted in the formation of micelles instead of P being entrapped in the bilayer of the liposomes. N did not reduce the phase transition temperature. All liposome suspensions used for DSC analysis were prepared in deionized water instead of sodium buffer to prevent undesired interactions. The samples were not extruded.

### Example 6: Liposomal Particle Size Distribution and Stability Assay

Liposomal size distribution was characterized via dynamic light scattering (DLS). Measurements were taken with disposable polystyrene sizing cuvettes containing the liposome suspensions. Reported DLS measurements are averages of 3 individually prepared liposome samples.

### Example 7: 2T-P and 2T-N Incorporation Efficiency Measurements

Parent compound and prodrug concentrations in liposomes were determined using UV-Vis spectrophotometry; (2T-P: 292 nm; 2T-N: 285 nm). PLL were prepared at varying concentrations between 0 mol % to 50 mol %. The lipid amounts remained equivalent for samples under 50 mol %. Each sample was extruded through a 200 nm pore membrane for a total of 11 passes. Prior and post extrusion liposomes were ruptured by dissolution in DMSO (liposome suspension/DMSO, 1:9, v/v). Samples were analyzed in a quartz cuvette. The following parameters were used: scan speed: 120 nm/min; bandwidth: 2 nm; integration time: 0.15 sec; data interval: 0.30 nm; start wavelength: 500 nm; end wavelength: 190 nm.

N and P (SCHEME 1) are hydrophobic compounds that have been altered to contain an amphiphilic tail (2T-P and 2T-N), altering their incorporation within liposomes, which can be characterized using UV-Vis. This technique measures the decreasing transmittance through a sample surface. The absorption of the liposomes at varying concentrations (after removal of unincorporated material) can be used to calculate how much of a drug has been incorporated into liposomes.

FIG. 7 PANEL A shows the drug incorporation within liposomes pre- and post-extrusion of 2T-P. FIG. 7 PANEL B shows the drug incorporation within liposomes pre- and post-extrusion if 2T-N. The liposomal drug incorporation limit was calculated to be <10 mol % for parent compounds, and on the order of 40 mol % for the prodrugs. Parent compound maximum measured loading were low (e.g., ˜11 mol % for P and ˜1.1 mol % for N).

Liposome solutions were passed through a membrane to remove unincorporated molecules. Samples were analyzed pre- and post-extrusion. Both prodrugs attained high loading capacities with 2T-N maintaining a 96% loading capacity (FIG. 6). Beyond 50 mol %, incorporation was unattainable due to inability to further extrude. The high standard deviations indicate instability of the liposomes resulted from unincorporated drug molecules within the liposomes.

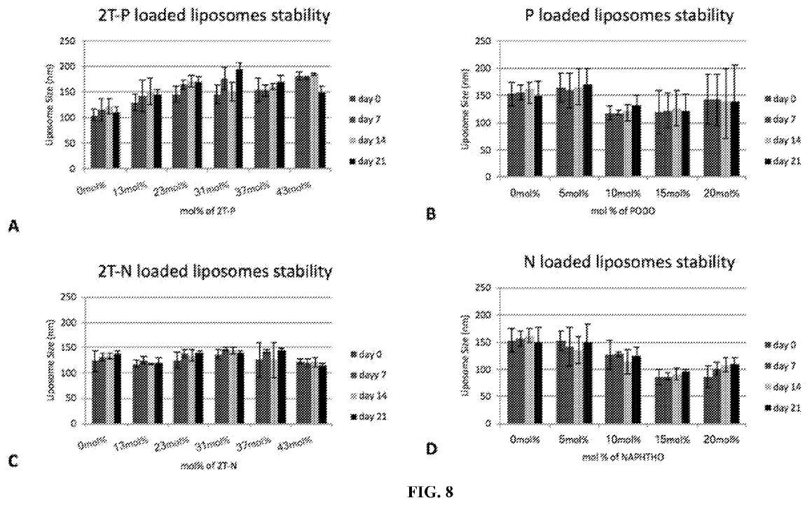

The size distribution of the parent compounds and prodrug loaded liposomes were monitored over time using dynamic light scattering (DLS). FIG. 8 PANEL A shows that 2T-P-loaded liposomes remained stable throughout a period of 3 weeks. FIG. 8 PANEL B shows that P-loaded liposomes were stable throughout a period of 3 weeks. FIG. 8 PANEL C shows that 2T-N-loaded liposomes remained stable throughout a period of 3 weeks. FIG. 8 PANEL D shows that N-loaded liposomes remained stable throughout a period of 3 weeks. Conjugating a potent drug to a phospholipid tail resulted in high loading efficiency, stability, retention, and targeted delivery of the liposomes.

The P-loaded liposome and N-loaded liposome concentrations reflected the initial drug amount, and not the final drug retention post-extrusion, which did not exceed 15 mol % (FIG. 7 PANEL A and FIG. 7 PANEL B).

The prodrug-loaded liposome size distributions demonstrated that the prodrugs remained within the liposomes with minimal leakage. Full saturation was observed when extrusion became impossible to complete due to the presence of excess unincorporated drug particles. Increasing both the loading efficiency and encapsulation stability minimized chemical degradation and allows for a feasible prodrug in lipophilic vehicles. Solubility was increased by adding hydrophilic tails and converting the hydrophobic drug to a lipophilic prodrug. Conversion resulted in high entrapment, low leakage, and chemical stability.

### Example 8: 2T-T Incorporation in Liposomes

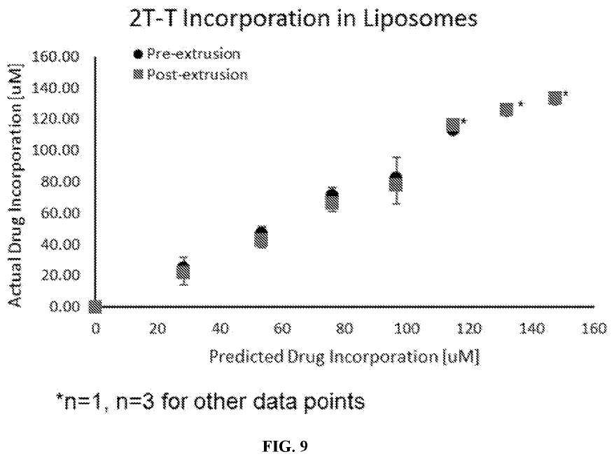

2T-T liposomes were produced with increasing concentrations of 2T-T, and passed through an extruder to remove any unincorporated 2T-T from the liposome solution. Liposome solutions were analyzed both pre- and post-extrusion using UV-vis spectroscopy with a scanning range of 200 nm-500 nm, 2 nm bandwidth, 30 second integration time, 0.5 nm data interval, and 100 nm/min scan speed. As can be seen in FIG. 9, and TABLE 1, the amount of 2T-T present post-extrusion is similar to pre-extrusion drug concentrations indicating minimal prodrug loss. Incorporation limits were analyzed up to 70 mol % (147 uM).

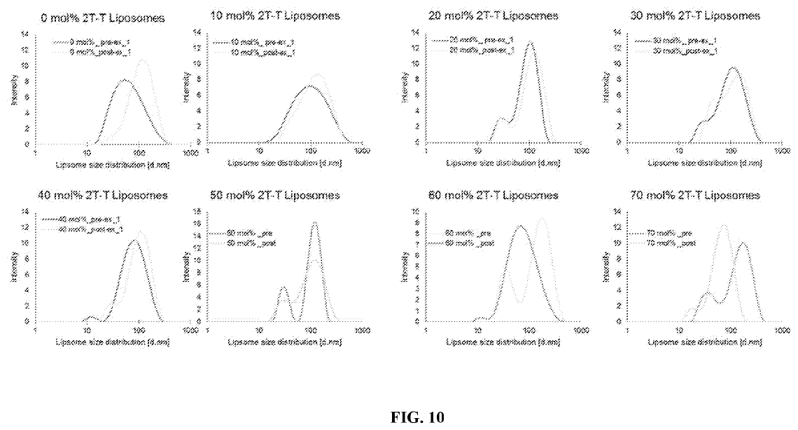

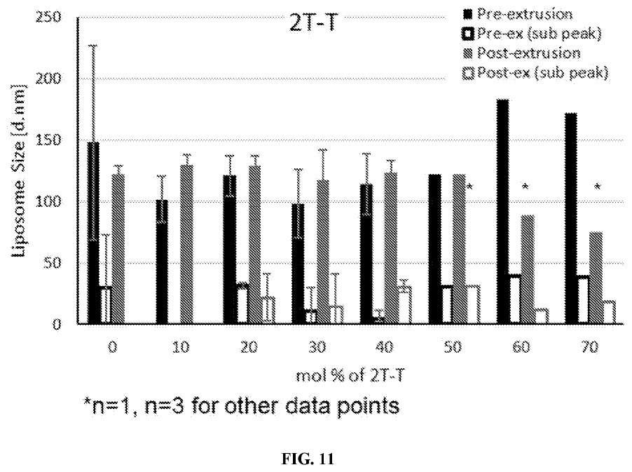

The size distribution of 2T-T liposomes were characterized via DLS measurement both pre- and post-extrusion. The standard deviations of liposome diameter were lower post extrusion. This result indicated an increased monodisperse population of liposomes. The size distributions of 2T-T liposomes at loaded with various concentrations of 2T-T can be seen in TABLE 2, and FIGS. 10-11.

TABLE 1

| Predicted Drug | Predicted Drug | Actual Drug | Actual Drug |
|---|---|---|---|
| Incorporation | Incorporation | Incorporation - | Incorporation - |
| [mol %] | [uM] | Pre [uM] | Post [uM] |
| 0 | 0 | 0 ± 0 | 0 ± 0 |
| 10 | 28.2 | 25.3 ± 6.6 | 22.1 ± 7.9 |
| 20 | 53.4 | 46.8 ± 4.8 | 42.8 ± 4.8 |
| 30 | 76.0 | 71.5 ± 5.1 | 66.2 ± 4.9 |
| 40 | 96.5 | 82.6 ± 13.1 | 77.9 ± 12.3 |
| 50* | 115.1 | 113.4 | 116.2 |
| 60* | 132.0 | 125.4 | 126.2 |
| 70* | 147.6 | 133.0 | 133.7 |

TABLE 2

| 2T-T mol % | Pre | PDI | Post | PDI |
|---|---|---|---|---|
| 0 | 148 ± 79 | 0.3 ± 0.1 | 122 ± 7 | 0.3 ± 0 |
| 10 | 102 ± 19 | 0.3 ± 0 | 130 ± 7 | 0.3 ± 0 |
| 20 | 121 ± 16 | 0.3 ± 0.1 | 129 ± 9 | 0.3 ± 0 |
| 30 | 98 ± 28 | 0.3 ± 0 | 118 ± 25 | 0.3 ± 0 |
| 40 | 114 ± 24 | 0.3 ± 0 | 123 ± 10 | 0.3 ± 0 |
| 50* | 122.4 | 0.3 | 122.4 | 0.3 |
| 60* | 182.9 | 0.5 | 88.76 | 0.3 |
| 70* | 172.1 | 0.4 | 75.43 | 0.3 |

PDI = polydispersity index

### Example 9: 2T-C Liposome Size Distribution

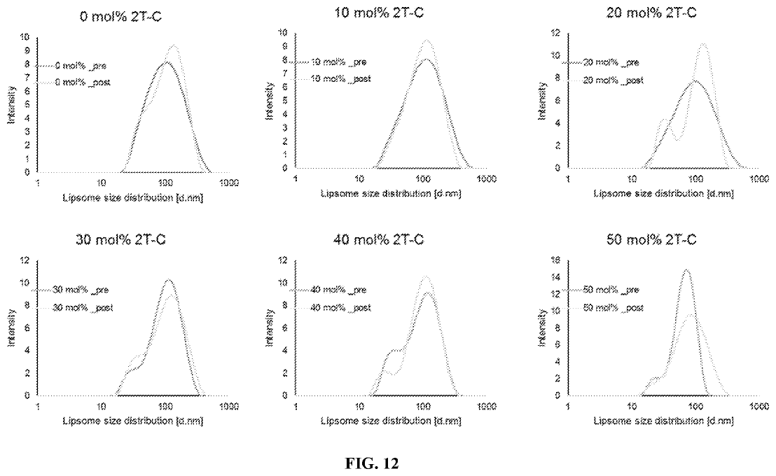

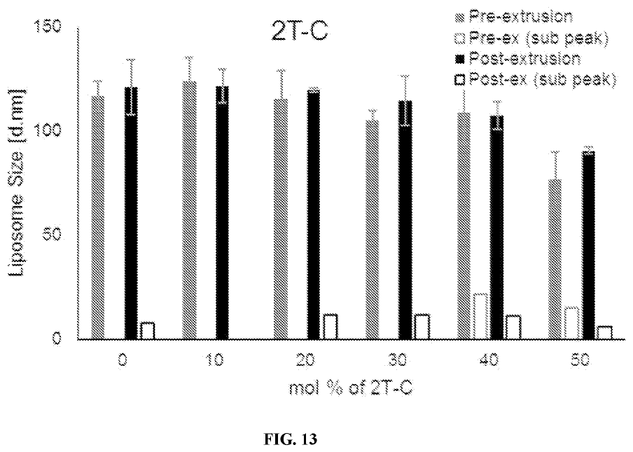

2T-C liposomes were produced with increasing concentrations of 2T-C and passed through an extruder to remove any unincorporated 2T-C from the liposome solution. The size distribution of 2T-C liposomes were characterized via DLS measurement both pre- and post-extrusion. The standard deviations of liposome diameter were lower post extrusion. This result indicated an increased monodisperse population of liposomes. The size distributions of 2T-C liposomes at loaded with various concentrations of 2T-C can be seen in TABLE 3, and FIGS. 12-13

TABLE 3

| 2T-C mol % | Pre | PDI | Post | PDI |
|---|---|---|---|---|
| 0 | 117 ± 7 | 0.3 ± 0 | 77 ± 13 | 0.3 ± 0 |
| 10 | 124 ± 11 | 0.3 ± 0 | 121 ± 8 | 0.3 ± 0 |
| 20 | 115 ± 14 | 0.3 ± 0 | 116 ± 1 | 0.3 ± 0 |
| 30 | 105 ± 4 | 0.3 ± 0 | 120 ± 12 | 0.3 ± 0 |
| 40 | 109 ± 15 | 0.3 ± 0 | 115 ± 7 | 0.3 ± 0 |
| 50 | 76.6 ± 13 | 0.4 ± 0.1 | 91 ± 2 | 0.3 ± 0 |

PDI = polydispersity index

### Example 10: Cell Culture

Human cervical cancer (ATCC S3) (HeLa) cells were cultured in DMEM supplemented with 10% FBS, 100 mg/L penicillin G, and 100 mg/L streptomycin. Human mammary carcinoma (MCF-7) cells were cultured in DMEM supplemented with 1.0 mM sodium pyruvate, 1% GlutaMax-1, 100 μg/mL penicillin, 100 μg/mL streptomycin, and 10% FBS. The cells were incubated at 37° C. in a humidified atmosphere with 5% CO₂. MCF10A cells were cultured in RPMI supplemented with 5% FBS, epidermal growth factor (20 ng/mL), hydrocortisone (0.5 ug/mL), cholera toxin (100 ng/mL), insulin (10 ug/mL), and PenStrep.

### Example 11: In Vitro Cytotoxicity of 2T-P and 2T-N Prodrug-Loaded Liposomes

Cells were seeded at 4,000 cells/well (HeLa, MCF7, MCF10a) in 96-well plates, and incubated for 24 hours at 37° C. and 5% CO₂. Following incubation, the media was replaced, and the cells were divided in parallel into a PLL treatment group and a parent compound treatment group. The cells were treated with a two-fold dilution series of PLL beginning at either a 2 vol % of the PLL suspension (prodrug/lipid, 0.2 mol %) or 1 μM of the parent compound. The PLL group positive controls were as follows: no treatment, empty liposomes (without drug, 2 vol %), and phenylarsine oxide (PAO). The parent compound group positive controls were as follows: no treatment, DMSO, and PAO. After 48 hours of incubation, 20 μL of 3-(4,5-dimethylthiazol-2-yl)-2,5-diphenyltetrazoliumbromide (MTT, 5 mg/mL) was added to each well, and the wells were incubated for 2 hours. The media was replaced with 100 μL of DMSO to dissolve the formazan crystals. Absorbance at 595 nm was measured using a microplate reader. The experiments were performed in four replicates and repeated twice.

The cytotoxicity of the prodrug-loaded liposomes (20 mol %) and parent compound were examined using a standard MTT assay for HeLa, MCF-7, and MCF10A cell lines. The 2T-N-loaded liposomes maintained potency, whereas the 2T-P-loaded liposomes lost activity. The reduction in activity resulted from steric hindrance within the 2T-P liposome structures. As a control, a normal breast cell line was studied to verify cancer cell differentiation when the cells were exposed to prodrug-loaded liposomes and the parent compound-loaded liposomes. The toxicity of the 2T-P-loaded liposomes was significantly reduced in all the cell lines tested compared to the parent compound P. The 2T-N-loaded liposomes maintained potency within HeLa cells (0.020 μM), but exhibited reduced potency in MCF7 (0.038 μM) cells. The potency of 2T-N-loaded liposomes in MCF10A cells was decreased substantially (1.105 μM). TABLE 4 shows the cell viability data of HeLa, MCF-7, and MCF10A cell lines after 48 hours of treatment with free parent compounds or prodrug-incorporated liposomes.

TABLE 4

| Cell Lines | P [μM] | 2T-P [μM] | N [μM] | 2T-N [μM] |
|---|---|---|---|---|
| HeLa | 0.009 ± 0.001 | 1.373 ± 0.044 | 0.005 ± 0.001 | 0.020 ± 0.002 |
| MCF7 | 0.009 ± 0.004 | 2.556 ± 0.126 | 0.003 ± 0.000 | 0.038 ± 0.007 |
| MCF10A | 0.014 ± 0.004 | 1.537 ± 0.130 | 0.005 ± 0.000 | 1.105 ± 0.131 |

### Example 12: In Vitro Cytotoxicity of 2T-T and 2T-C Prodrug-Loaded Liposomes

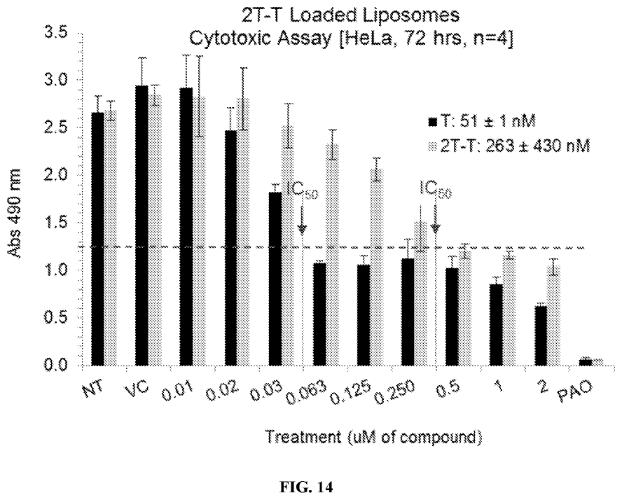

To assess the cytotoxicity of 2T-T prodrug-loaded liposomes, HeLa cells were exposed to different concentrations of topotecan or 2T-T loaded liposomes for 72 hours. After the 72 hour incubation period, cell viability was assessed via an MTT assay. As can be seen in FIG. 14, the IC₅₀ of free topotecan was found to be 51±1 nM, while the IC₅₀ of 2T-T liposomes was 263±430 nM.

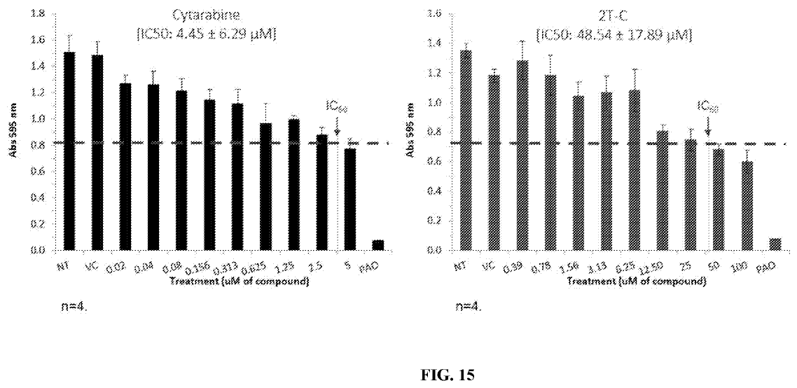

To assess the cytotoxicity of 2T-C prodrug-loaded liposomes, HeLa cells were exposed to different concentrations of cytarabine or 2T-C(NH₂ attachment) loaded liposomes for 48 hours. After the 48 hour incubation period, cell viability was assessed via a MTT assay. As can be seen in FIG. 15, the IC₅₀ of free cytarabine was found to be 4.45±6.29 μM, while the IC₅₀ of 2T-C liposomes was 48.54±17.89 μM.

### Example 13: In Vitro Ultrasound

Microbubbles generated with 0 mol % and 20 mol % prodrug concentrations were purified by centrifugation at 0.3 ref for 10 mins. The supernatant (microbubbles) and liposomes were separated. The supernatant was incubated in culture media with serum for 12 hrs, and spun by centrifuge again. HeLa cells were seeded at 120,000 cells/well onto coverslips in 6-well plates. Once confluent cells were observed, the coverslips were setup up into a cell-plate chamber in contact with 2 μL microbubbles: 3 mL media. The chambers were sealed with another coverslip, placed in the ultrasound chamber, and exposed to 18 pulses of ultrasound. The coverslips were then immediately washed 3 times with media to remove excess microbubbles. After 20 hours of incubation, the cell plates were imaged in bright field using a microscope. No treatment was used as a control, and inverted cell-plates were used to facilitate contact with 0 mol % and 20 mol % prodrug-loaded microbubbles in the absence of ultrasound exposure.

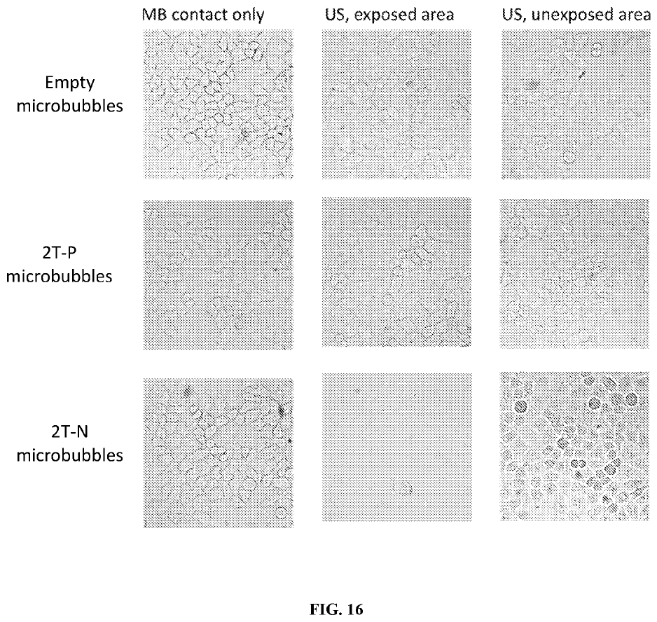

The prodrugs were incorporated into microbubbles for focused drug release using ultrasound. FIG. 16 shows in vitro ultrasound-triggered delivery of prodrug-loaded microbubbles. The microbubbles were purified, incubated in serum, and purified again prior to testing. The microbubbles did not dissociate with the serum. The in vitro cytotoxicity of localized microbubble delivery using ultrasound validated the localized and ultrasound-triggered delivery of 2T-N-infused microbubbles. Empty microbubbles and prodrug-loaded microbubbles were placed in contact with HeLa cells for ultrasound-free controls to confirm localization using ultrasound (FIG. 16, Left column). Empty and 2T-P-loaded microbubble and ultrasound-treated cells remained confluent in the ultrasound-exposed and ultrasound-unexposed areas. 2T-N-loaded microbubble and ultrasound-treated cells diminished in the ultrasound-exposed area, but remained confluent in the ultrasound-unexposed areas.

### Example 14: Enzymatic Assay

Enzymatic cleavage was qualitatively measured using a spectrofluorophotometer. Porcine liver esterase was diluted in 1×PBS from a concentrated stock solution to 1.2×10⁻⁷ M and stored at −20° C. At time zero, 100 μL of empty or PLLs were placed in a 100 μL cuvette and 5 μL of esterase was added. The solution was immediately measured in a spectrofluorophotometer. The sample was measured again at 60 minutes. The following parameters were used: medium scanning speed, 2 second response time, 1 nm sampling interval, 3 nm excitation slit width, 20 nm emission slit width, high sensitivity, an excitation wavelength of 250 nm, and an emission range of 280 nm-600 nm.

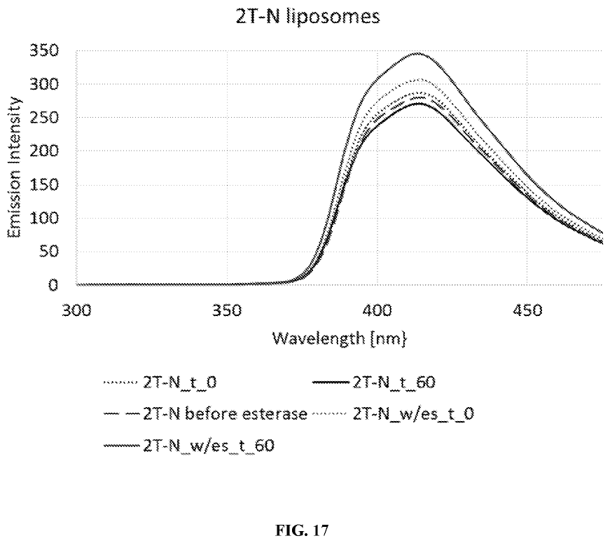

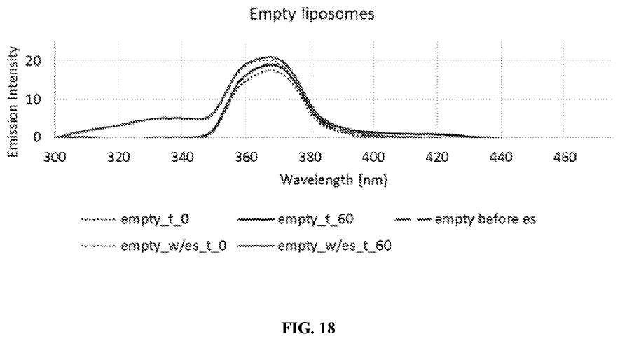

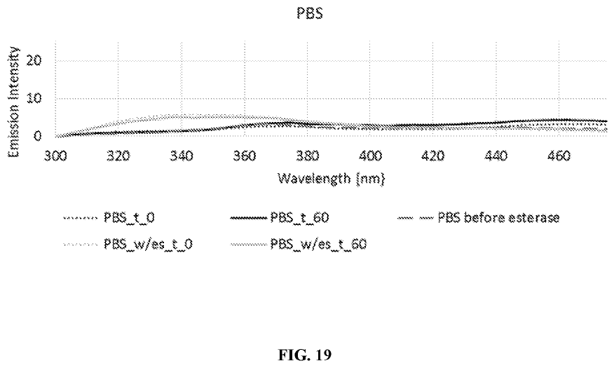

FIG. 17 shows the fluorescence spectra of 2T-N-loaded liposomes at different time points with and without treatment with porcine liver esterase. TABLE 5 shows the changes in size of empty and 2T-N liposomes following esterase treatment. FIG. 18 shows the fluorescence spectra of empty liposomes at different time points with and without treatment with porcine liver esterase. FIG. 19 shows the fluorescence spectra of a PBS solution at different time points with and without treatment with porcine liver esterase.

TABLE 5

| | Liposome size | |
|---|---|---|
| | (d · nm) | PDI |
| Empty | 116.87 ± 2.4 | 0.31 ± 0.06 |
| Empty with esterase | 121.97 ± 2.65 | 0.27 ± 0.01 |
| 2T-N | 141.47 ± 4.48 | 0.27 ± 0.00 |
| 2T-N with esterase | 151.23 ± 15.62 | 0.31 ± 0.03 |

### EMBODIMENTS

The following non-limiting embodiments provide illustrative examples of the invention, but do not limit the scope of the invention.

Embodiment 1. A lipid-based carrier comprising: a) a surface layer, wherein the surface layer comprises a prodrug, wherein the prodrug comprises a therapeutic agent covalently conjugated to a phospholipid; and b) a core, wherein the surface layer surrounds the core.

Embodiment 2. The lipid-based carrier of embodiment 1, wherein the lipid-based carrier is a microbubble.

Embodiment 3. The lipid-based carrier of embodiments 1 or 2, wherein the surface layer is a lipid monolayer.

Embodiment 4. The lipid-based carrier of any one of embodiments 1-3, wherein the core is a gas.

Embodiment 5. The lipid-based carrier of embodiment 4, wherein the gas is sulfur hexafluoride (SF₆).

Embodiment 6. The lipid-based carrier of embodiment 1 or 3, wherein the core is a solid.

Embodiment 7. The lipid-based carrier of embodiment 6, wherein the solid is a metal.

Embodiment 8. The lipid-based carrier of embodiment 6, wherein the solid is a semiconductor.

Embodiment 9. The lipid-based carrier of embodiment 1 or 3, wherein the core is a liquid.

Embodiment 10. The lipid-based carrier of any one of embodiments 1-3, wherein the core is an organic material.

Embodiment 11. The lipid-based carrier of any one of embodiments 1-3, wherein the core is an inorganic material.

Embodiment 12. The lipid-based carrier of embodiment 1 or 3, wherein the core is an aqueous solution.

Embodiment 13. The lipid-based carrier of any one of embodiments 1 or 4-12, wherein the lipid-based carrier is a liposome.

Embodiment 14. The lipid-based carrier of any one of embodiments 1, 2, or 4-13, wherein the surface layer is a lipid bilayer.

Embodiment 15. The lipid-based carrier of any one of embodiments 1-14, wherein the lipid-based carrier has a diameter of about 70 nm to about 900 nm.

Embodiment 16. The lipid-based carrier of any one of embodiments 1-15, wherein the prodrug is present in an amount of about 1 mol % to about 100 mol %.

Embodiment 17. The lipid-based carrier of any one of embodiments 1-16, wherein the phospholipid is a two-tailed phospholipid.

Embodiment 18. The lipid-based carrier of any one of embodiments 1-17, wherein the phospholipid comprises a hydrophobic tail comprising about 10 carbon atoms to about 24 carbon atoms.

Embodiment 19. The lipid-based carrier of any one of embodiments 1-18, wherein the phospholipid comprises a hydrophobic tail comprising about 16 carbon atoms.

Embodiment 20. The lipid-based carrier of any one of embodiments 1-19, wherein the therapeutic agent is an anticancer agent.

Embodiment 21. The lipid-based carrier of embodiment 20, wherein the anticancer agent is topotecan.

Embodiment 22. The lipid-based carrier of embodiment 20, wherein the anticancer agent is cytarabine.

Embodiment 23. The lipid-based carrier of any one of embodiments 1-19, wherein the therapeutic agent is a compound of the formula:

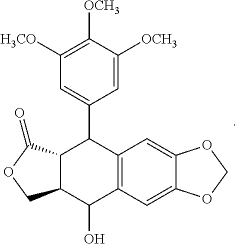 [SMILES: COc1cc(C2c3cc4c(cc3C(O)[C@H]3COC(=O)[C@H]23)OCO4)cc(OC)c1OC]

Embodiment 24. The lipid-based carrier of any one of embodiments 1-19, wherein the therapeutic agent is a compound of the formula:

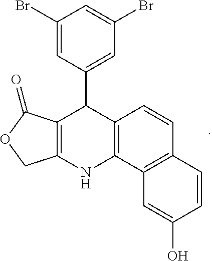 [SMILES: O=C1OCC2=C1C(c1cc(Br)cc(Br)c1)c1ccc3ccc(O)cc3c1N2]

Embodiment 25. The lipid-based carrier of any one of embodiments 1-19, wherein the therapeutic agent is an anti-viral agent.

Embodiment 26. The lipid-based carrier of any one of embodiments 1-19, wherein the therapeutic agent is an anti-bacterial agent.

Embodiment 27. The lipid-based carrier of any one of embodiments 1-19, wherein the therapeutic agent is a neurotransmitter.

Embodiment 28. The lipid-based carrier of any one of embodiments 1-19, wherein the therapeutic agent is a protein.

Embodiment 29. The lipid-based carrier of any one of embodiments 1-19, wherein the therapeutic agent is a biologic.

Embodiment 30. The lipid-based carrier of any one of embodiments 1-19, wherein the therapeutic agent is gemcitabine.

Embodiment 31. The lipid-based carrier of any one of embodiments 1-19, wherein the therapeutic agent is a chelating agent.

Embodiment 32. The lipid-based carrier of any one of embodiments 1-31, wherein the surface layer further comprises DPPC.

Embodiment 33. The lipid-based carrier of any one of embodiments 1-32, wherein the surface layer further comprises DPPA.

Embodiment 34. The lipid-based carrier of any one of embodiments 1-33, wherein the surface layer further comprises DSPE-PEG2000.

Embodiment 35. The lipid-based carrier of any one of embodiments 1-34, wherein the surface layer further comprises DSPE-PEG5000.

Embodiment 36. The lipid-based carrier of any one of embodiments 1-35, wherein the phospholipid is an activated phospholipid.

Embodiment 37. The lipid-based carrier of embodiment 36, wherein the activated phospholipid is a Glu-phospholipid.

Embodiment 38. The lipid-based carrier of embodiment 36, wherein the activated phospholipid is a NHS-phospholipid.

Embodiment 39. The lipid-based carrier of embodiment 36, wherein the activated phospholipid is a PDP-phospholipid.

Embodiment 40. The lipid-based carrier of embodiment 36, wherein the activated phospholipid is a MAL-phospholipid.

Embodiment 41. The lipid-based carrier of embodiment 36, wherein the activated phospholipid is a NBD-phospholipid.

Embodiment 42. The lipid-based carrier of embodiment 37, wherein the Glu-phospholipid is DPPE-Glu.

Embodiment 43. The lipid based carrier of any one of embodiments 1-42, wherein the therapeutic agent is covalently conjugated to the phospholipid by an ester bond.

Embodiment 44. The lipid based carrier of any one of embodiments 1-42, wherein the therapeutic agent is covalently conjugated to the phospholipid by an amide bond.

Embodiment 45. A method of treating a condition, the method comprising administering to a subject in need thereof a therapeutically-effective amount of a lipid-based carrier, the lipid-based carrier comprising: a) a surface layer, wherein the surface layer comprises a prodrug, wherein the prodrug comprises a therapeutic agent covalently conjugated to a phospholipid; and b) a core, wherein the surface layer surrounds the core.

Embodiment 46. The method of embodiment 45, wherein the lipid-based carrier is a microbubble.

Embodiment 47. The method of embodiment 45 or 46, wherein the surface layer is a lipid monolayer.

Embodiment 48. The method of any one of embodiments 45-47, wherein the core is a gas.

Embodiment 49. The method of embodiment 48, wherein the gas is sulfur hexafluoride (SF₆).

Embodiment 50. The method of embodiment 45 or 47, wherein the core is a solid.

Embodiment 51. The method of embodiment 50, wherein the solid is a metal.

Embodiment 52. The method of embodiment 50, wherein the solid is a semiconductor.

Embodiment 53. The method of embodiment 45 or 47, wherein the core is a liquid.

Embodiment 54. The method of any one of embodiments 45-47, wherein the core is an organic material.

Embodiment 55. The method of any one of embodiments 45-47, wherein the core is an inorganic material.

Embodiment 56. The method of embodiment 45 or 47, wherein the core is an aqueous solution.

Embodiment 57. The method of any one of embodiments 45 or 48-56, wherein the lipid-based carrier is a liposome.

Embodiment 58. The method of any one of embodiments 45, 46, or 48-57, wherein the surface layer is a lipid bilayer.

Embodiment 59. The method of any one of embodiments 45-58, wherein the lipid-based carrier has a diameter of about 70 nm to about 900 nm.

Embodiment 60. The method of any one of embodiments 45-59, wherein the prodrug is present in an amount of about 1 mol % to about 100 mol %.

Embodiment 61. The method of any one of embodiments 45-60, wherein the phospholipid is a two-tailed phospholipid.

Embodiment 62. The method of any one of embodiments 45-61, wherein the phospholipid comprises a hydrophobic tail comprising about 10 carbon atoms to about 24 carbon atoms.

Embodiment 63. The method of any one of embodiments 45-62, wherein the phospholipid comprises a hydrophobic tail comprising about 16 carbon atoms.

Embodiment 64. The method of any one of embodiments 45-63, wherein the therapeutic agent is an anticancer agent.

Embodiment 65. The method of embodiment 64, wherein the anticancer agent is topotecan.

Embodiment 66. The method of embodiment 64, wherein the anticancer agent is cytarabine.

Embodiment 67. The method of any one of embodiment 45-63, wherein the therapeutic agent is a compound of the formula:

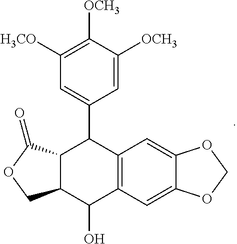 [SMILES: COc1cc(C2c3cc4c(cc3C(O)[C@H]3COC(=O)[C@H]23)OCO4)cc(OC)c1OC]

Embodiment 68. The method of any one of embodiment 45-63, wherein the therapeutic agent is a compound of the formula:

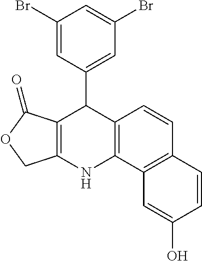 [SMILES: O=C1OCC2=C1C(c1cc(Br)cc(Br)c1)c1ccc3ccc(O)cc3c1N2]

Embodiment 69. The method of any one of embodiments 45-63, wherein the therapeutic agent is an anti-viral agent.

Embodiment 70. The method of any one of embodiments 45-63, wherein the therapeutic agent is an anti-bacterial agent.

Embodiment 71. The method of any one of embodiments 45-63, wherein the therapeutic agent is a neurotransmitter.

Embodiment 72. The method of any one of embodiments 45-63, wherein the therapeutic agent is a protein.

Embodiment 73. The method of any one of embodiments 45-63, wherein the therapeutic agent is a biologic.

Embodiment 74. The method of any one of embodiments 45-63, wherein the therapeutic agent is gemcitabine.

Embodiment 75. The method of any one of embodiments 45-63, wherein the therapeutic agent is a chelating agent.

Embodiment 76. The method of any one of embodiments 45-75, wherein the surface layer further comprises DPPC.

Embodiment 77. The method of any one of embodiments 45-76, wherein the surface layer further comprises DPPA.

Embodiment 78. The method of any one of embodiments 45-77, wherein the surface layer further comprises DSPE-PEG2000.

Embodiment 79. The method of any one of embodiments 45-78, wherein the surface layer further comprises DSPE-PEG5000.

Embodiment 80. The method of any one of embodiments 45-79, wherein the phospholipid is an activated phospholipid.

Embodiment 81. The method of embodiment 80, wherein the activated phospholipid is a Glu-phospholipid.

Embodiment 82. The method of embodiment 80, wherein the activated phospholipid is a NHS-phospholipid.

Embodiment 83. The method of embodiment 80, wherein the activated phospholipid is a PDP-phospholipid.

Embodiment 84. The method of embodiment 80, wherein the activated phospholipid is a MAL-phospholipid.

Embodiment 85. The method of embodiment 80, wherein the activated phospholipid is a NBD-phospholipid.

Embodiment 86. The method of embodiment 81, wherein the Glu-phospholipid is DPPE-Glu.

Embodiment 87. The method of any one of embodiments 45-86, wherein the therapeutic agent is covalently conjugated to the phospholipid by an ester bond.

Embodiment 88. The method of any one of embodiments 45-86, wherein the therapeutic agent is covalently conjugated to the phospholipid by an amide bond

Embodiment 89. The method of any one of embodiments 45-89, further comprising applying an extracorporeal trigger to the subject.

Embodiment 90. The method of embodiment 89, wherein the extracorporeal trigger is an ultrasound frequency.

Embodiment 91. The method of embodiment 90, wherein the ultrasound frequency is from about 1 MHz to about 20 MHz.

Embodiment 92. The method of embodiment 89, wherein the extracorporeal trigger is light.

Embodiment 93. The method of embodiment 92, wherein the light has a wavelength of about 400 nm to about 1400 nm.

Embodiment 94. The method of embodiment 89, wherein the extracorporeal trigger is an electric field.

Embodiment 95. The method of embodiment 89, wherein the extracorporeal trigger is a magnetic field.

Embodiment 96. The method of embodiment 95, wherein the magnetic field has a strength of about 0.2 T to about 7 T.

Embodiment 97. The method of any one of embodiments 89-96, wherein the extracorporeal trigger is applied in pulses.

Embodiment 98. The method of any one of embodiments 45-97, wherein the administration is intravenous.

Embodiment 99. The method of any one of embodiments 45-97, wherein the administration is intratumoral.

Embodiment 100. The method of any one of embodiments 45-97, wherein the administration is subcutaneous.

Embodiment 101. The method of any one of embodiments 45-97, wherein the administration is intra-arterial.

What is claimed is:

1. A lipid-based carrier comprising:
a) a surface layer, wherein the surface layer comprises a prodrug, wherein the prodrug comprises a therapeutic agent covalently conjugated to a phospholipid; and
b) a core, wherein the surface layer surrounds the core.

2. The lipid-based carrier of claim 1, wherein the lipid-based carrier is a microbubble.

3. The lipid-based carrier of claim 1, wherein the surface layer is a lipid monolayer.

4. The lipid-based carrier of claim 1, wherein the core is a gas.

5. The lipid-based carrier of claim 4, wherein the gas is sulfur hexafluoride (SF₆).

6. The lipid-based carrier of claim 1, wherein the core is a solid.

7. The lipid-based carrier of claim 6, wherein the solid is a metal.

8. The lipid-based carrier of claim 6, wherein the solid is a semiconductor.

9. The lipid-based carrier of claim 1, wherein the core is a liquid.

10. The lipid-based carrier of claim 1, wherein the core is an organic material.

11. The lipid-based carrier of claim 1, wherein the core is an inorganic material.

12. The lipid-based carrier of claim 1, wherein the core is an aqueous solution.

13. The lipid-based carrier of claim 1, wherein the lipid-based carrier is a liposome.

14. The lipid-based carrier of claim 1, wherein the surface layer is a lipid bilayer.

15. The lipid-based carrier of claim 1, wherein the lipid-based carrier has a diameter of about 70 nm to about 900 nm.

16. The lipid-based carrier of claim 1, wherein the prodrug is present in an amount of about 1 mol % to about 100 mol %.

17. The lipid-based carrier of claim 1, wherein the phospholipid is a two-tailed phospholipid.

18. The lipid-based carrier of claim 1, wherein the phospholipid comprises a hydrophobic tail comprising about 10 carbon atoms to about 24 carbon atoms.

19. The lipid-based carrier of claim 1, wherein the phospholipid comprises a hydrophobic tail comprising about 16 carbon atoms.

20. The lipid-based carrier of claim 1, wherein the therapeutic agent is an anticancer agent.

21. The lipid-based carrier of claim 20, wherein the anticancer agent is topotecan.

22. The lipid-based carrier of claim 20, wherein the anticancer agent is cytarabine.

23. The lipid-based carrier of claim 1, wherein the therapeutic agent is a compound of the formula:

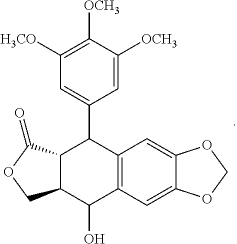 [SMILES: COc1cc(C2c3cc4c(cc3C(O)[C@H]3COC(=O)[C@H]23)OCO4)cc(OC)c1OC]

24. The lipid-based carrier of claim 1, wherein the therapeutic agent is a compound of the formula:

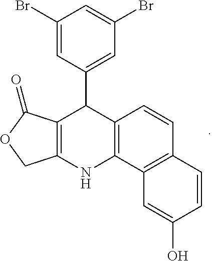 [SMILES: O=C1OCC2=C1C(c1cc(Br)cc(Br)c1)c1ccc3ccc(O)cc3c1N2]

25. The lipid-based carrier of claim 1, wherein the therapeutic agent is an anti-viral agent.

26. The lipid-based carrier of claim 1, wherein the therapeutic agent is an anti-bacterial agent.

27. The lipid-based carrier of claim 1, wherein the therapeutic agent is a neurotransmitter.

28. The lipid-based carrier of claim 1, wherein the therapeutic agent is a protein.

29. The lipid-based carrier of claim 1, wherein the therapeutic agent is a biologic.

30. The lipid-based carrier of claim 1, wherein the therapeutic agent is gemcitabine.

31. The lipid-based carrier of claim 1, wherein the therapeutic agent is a chelating agent.

32. The lipid-based carrier of claim 1, wherein the surface layer further comprises DPPC.

33. The lipid-based carrier of claim 1, wherein the surface layer further comprises DPPA.

34. The lipid-based carrier of claim 1, wherein the surface layer further comprises DSPE-PEG2000.

35. The lipid-based carrier of claim 1, wherein the surface layer further comprises DSPE-PEG5000.

36. The lipid-based carrier of claim 1, wherein the phospholipid is an activated phospholipid.

37. The lipid-based carrier of claim 36, wherein the activated phospholipid is a Glu-phospholipid.

38. The lipid-based carrier of claim 36, wherein the activated phospholipid is a NHS-phospholipid.

39. The lipid-based carrier of claim 36, wherein the activated phospholipid is a PDP-phospholipid.

40. The lipid-based carrier of claim 36, wherein the activated phospholipid is a MAL-phospholipid.

41. The lipid-based carrier of claim 36, wherein the activated phospholipid is a NBD-phospholipid.

42. The lipid-based carrier of claim 37, wherein the Glu-phospholipid is DPPE-Glu.

43. The lipid-based carrier of claim 1, wherein the therapeutic agent is covalently conjugated to the phospholipid by an ester bond.

44. The lipid based carrier of claim 1, wherein the therapeutic agent is covalently conjugated to the phospholipid by an amide bond.

45. A method of treating a condition, the method comprising administering to a subject in need thereof a therapeutically-effective amount of a lipid-based carrier, the lipid-based carrier comprising:
a) a surface layer, wherein the surface layer comprises a prodrug, wherein the prodrug comprises a therapeutic agent covalently conjugated to a phospholipid; and
b) a core, wherein the surface layer surrounds the core.

46. The method of claim 45, wherein the lipid-based carrier is a microbubble.

47. The method of claim 45, wherein the surface layer is a lipid monolayer.

48. The method of claim 45, wherein the core is a gas.

49. The method of claim 48, wherein the gas is sulfur hexafluoride (SF₆).

50. The method of claim 45, wherein the core is a solid.

51. The method of claim 50, wherein the solid is a metal.

52. The method of claim 50, wherein the solid is a semiconductor.

53. The method of claim 45, wherein the core is a liquid.

54. The method of claim 45, wherein the core is an organic material.

55. The method of claim 45, wherein the core is an inorganic material.

56. The method of claim 45, wherein the core is an aqueous solution.

57. The method of claim 45, wherein the lipid-based carrier is a liposome.

58. The method of claim 45, wherein the surface layer is a lipid bilayer.

59. The method of claim 45, wherein the lipid-based carrier has a diameter of about 70 nm to about 900 nm.

60. The method of claim 45, wherein the prodrug is present in an amount of about 1 mol % to about 100 mol %.

61. The method of claim 45, wherein the phospholipid is a two-tailed phospholipid.

62. The method of claim 45, wherein the phospholipid comprises a hydrophobic tail comprising about 10 carbon atoms to about 24 carbon atoms.

63. The method of claim 45, wherein the phospholipid comprises a hydrophobic tail comprising about 16 carbon atoms.

64. The method of claim 45, wherein the therapeutic agent is an anticancer agent.

65. The method of claim 64, wherein the anticancer agent is topotecan.

66. The method of claim 64, wherein the anticancer agent is cytarabine.

67. The method of claim 45, wherein the therapeutic agent is a compound of the formula:

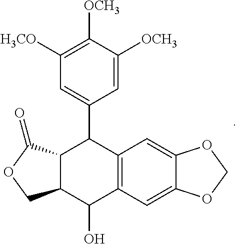 [SMILES: COc1cc(C2c3cc4c(cc3C(O)[C@H]3COC(=O)[C@H]23)OCO4)cc(OC)c1OC]

68. The method of claim 45, wherein the therapeutic agent is a compound of the formula:

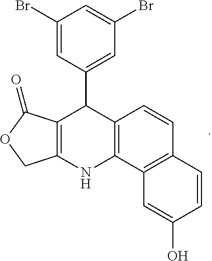 [SMILES: O=C1OCC2=C1C(c1cc(Br)cc(Br)c1)c1ccc3ccc(O)cc3c1N2]

69. The method of claim 45, wherein the therapeutic agent is an anti-viral agent.

70. The method of claim 45, wherein the therapeutic agent is an anti-bacterial agent.

71. The method of claim 45, wherein the therapeutic agent is a neurotransmitter.

72. The method of claim 45, wherein the therapeutic agent is a protein.

73. The method of claim 45, wherein the therapeutic agent is a biologic.

74. The method of claim 45, wherein the therapeutic agent is gemcitabine.

75. The method of claim 45, wherein the therapeutic agent is a chelating agent.

76. The method of claim 45, wherein the surface layer further comprises DPPC.

77. The method of claim 45, wherein the surface layer further comprises DPPA.

78. The method of claim 45, wherein the surface layer further comprises DSPE-PEG2000.

79. The method of claim 45, wherein the surface layer further comprises DSPE-PEG5000.

80. The method of claim 45, wherein the phospholipid is an activated phospholipid.

81. The method of claim 80, wherein the activated phospholipid is a Glu-phospholipid.

82. The method of claim 80, wherein the activated phospholipid is a NHS-phospholipid.

83. The method of claim 80, wherein the activated phospholipid is a PDP-phospholipid.

84. The method of claim 80, wherein the activated phospholipid is a MAL-phospholipid.

85. The method of claim 80, wherein the activated phospholipid is a NBD-phospholipid.

86. The method of claim 81, wherein the Glu-phospholipid is DPPE-Glu.

87. The method of claim 45, wherein the therapeutic agent is covalently conjugated to the phospholipid by an ester bond.

88. The method of claim 45, wherein the therapeutic agent is covalently conjugated to the phospholipid by an amide bond.

89. The method of claim 45, further comprising applying an extracorporeal trigger to the subject.

90. The method of claim 89, wherein the extracorporeal trigger is an ultrasound frequency.

91. The method of claim 90, wherein the ultrasound frequency is from about 1 MHz to about 20 MHz.

92. The method of claim 89, wherein the extracorporeal trigger is light.

93. The method of claim 92, wherein the light has a wavelength of about 400 nm to about 1400 nm.

94. The method of claim 89, wherein the extracorporeal trigger is an electric field.

95. The method of claim 89, wherein the extracorporeal trigger is a magnetic field.

96. The method of claim 95, wherein the magnetic field has a strength of about 0.2 T to about 7 T.

97. The method of claim 89, wherein the extracorporeal trigger is applied in pulses.

98. The method of claim 45, wherein the administration is intravenous.

99. The method of claim 45, wherein the administration is intratumoral.

100. The method of claim 45, wherein the administration is subcutaneous.

101. The method of claim 45, wherein the administration is intra-arterial.
